(19) United States
(12) Patent Application Publication
(10) Pub. No.: US 2026/0102196 A1
(43) Pub. Date: Apr. 16, 2026

(54) CATHETER, SYSTEM, AND METHOD FOR SELECTIVE ABLATION IN THE MUCOSA AND SUBMUCOSA OF THE GASTROINTESTINAL TRACT

(71) Applicant: Agil Therapeutics, Inc., San Francisco, CA (US)

(72) Inventors: Timothy D. HOLLAND, Los Gatos, CA (US); Peter GARCIA-MEZA, San Francisco, CA (US)

(21) Appl. No.: 19/115,241

(22) PCT Filed: Sep. 26, 2023
(86) PCT No.: PCT/US2023/075176

§ 371 Date: Mar. 25, 2025

Related U.S. Application Data
(60) Provisional application No. 63/410,171, filed on Sep. 26, 2022.

(51) Int. Cl. A61B 18/02; A61B 18/00; A61B 90/00

(52) U.S. Cl. CPC A61B 18/02; A61B 90/06; A61B 2018/00184; A61B 2018/00488; A61B 2018/00494; A61B 2018/005; A61B 2018/00577; A61B 2018/00642; A61B 2018/00744; A61B 2018/00773; A61B 2018/0091; A61B 2018/0212; A61B 2018/0231; A61B 2018/0268; A61B 2090/064

(57) ABSTRACT

Catheters, systems, and methods for cryogenic ablation of gastrointestinal tract tissue are described. Catheter includes a probe and a cryogenic supply assembly housed within the probe. The cryogenic supply assembly includes a sprayer orifice assembly having a sprayer tube and a seal chamber component, and a cryogenic supply line within the sprayer orifice assembly. The sprayer tube includes one or more sprayer orifices for delivery of a cryogenic fluid from the cryogenic supply line to the probe. The seal chamber component includes a seal chamber associated with each of the one or more sprayer orifices and forms a fluidic conduit between the cryogenic supply line and the one or more sprayer orifices through which the cryogenic fluid flows to the probe. The cryogenic supply assembly is configured so that the cryogenic supply line and the sprayer tube move either axially independent of one another or axially together as one unit.

### CROSS-REFERENCE TO RELATED APPLICATIONS

This application claims priority benefit of U.S. Provisional Patent Application No. 63/410,171, filed Sep. 26, 2022, the disclosure of which is hereby incorporated by reference herein in its entirety.

### FIELD OF THE DISCLOSURE

The present disclosure generally relates to medical devices (catheters), systems, and methods for their use for selectively ablating of the mucosa and submucosa in the gastrointestinal tract, including, inter alia, for the treatment of Type 2 Diabetes, obesity, and other metabolic conditions.

### BACKGROUND OF THE DISCLOSURE

Type 2 diabetes is a chronic condition that affects how the body metabolizes glucose. The effects of diabetes are staggering (over 10% of the US population has diabetes) to include being at higher risk of cardiovascular disease, retinopathy/macular oedema/glaucoma, nephropathy, neuropathy, and other conditions (1). Conventional medical treatment of type 2 diabetes only partially achieves adequate glycemic control and a reduction in cardiovascular risk (2). Thus, other approaches are needed.

Although developed as a weight reduction therapy, gastric-bypass surgery such as RYGB (Roux-en-Y bypass) has been reported to improve or eliminate type 2 diabetes in 70 to 80% of postoperative patients (3). The rapidity of the correction of glucose concentration in the blood and good glycemic regulation might be a result of the secondary alteration in incretin (hormonal) signals from the antrum, duodenum, and the proximal jejunum to the pancreatic islets (4). RYGB has shown to decrease or eliminate hormonal or neural signal(s) that normally would emanate from the stomach, pylorus, duodenum, or jejunum upon exposure to nutrient passage through the gastrointestinal tract. While effective, RYGB carries a substantial risk of surgical morbidity and mortality. Endoscopic interventions that combine the remarkable effectiveness of RYGB while minimizing associated risks would be a highly desirable addition to available treatments.

A technique that mimics RYGB is the DJBS (duodenal-jejunal bypass sleeve). Unlike RYGB, the DJBS (U.S. Pat. No. 7,819,836) is a fully reversible, nonsurgical procedure while still leveraging an important aspect of RYGB (eliminating nutrient exposure to the duodenum and jejunum). The clinical effects of improved glycemic control of the DJBL provide more evidence for the role of the duodenum in the interplay of obesity, metabolic syndrome, and type 2 diabetes. However, based on the rather high adverse event rate of the DJBL, this technique will not be likely adopted (5).

An additional nonsurgical avenue of investigation is ablation of the wall of one or more of the same organs of the gastrointestinal tract. Ablation could result in a similar absence of hormonal or neural signals as in bariatric surgery. Ablation of the duodenum is of particular interest given that the duodenum is recognized as a metabolic signaling center that seems to play a role in regulating insulin action and, therefore, insulin resistance (6).

One specific technique is hydrothermal ablation (U.S. Pat. No. 9,757,535) of the mucosa of the duodenum or DMR (duodenal mucosal resurfacing). The desired ablation is limited to the superficial intestinal mucosa leaving any deeper structures untouched. The goal with DMR is restoration to a normal mucosal interface in the duodenum (7). DMR has been found to be feasible and safe in that it elicited glycemic improvement in sub optimally controlled T2D patients using oral glucose-lowering medication (8). While showing promise, DMR has shown adverse events such as increased postoperative pain and usability shortcomings such as the need for fluoroscopy which could limit widespread adoption.

An additional ablative technique uses a laser to selectively target the submucosa of the duodenal wall and neural structures (nerves cells as ganglions, plexuses, axon, etc.) within. The proposed technique EGM (U.S. Pat. No. 10,575,904) or endoscopic glycemic management targets a large portion of the duodenum as nerves of the duodenum travel throughout the submucosal layer of the duodenum. The application of a laser does address usability shortcomings of hydrothermal resurfacing of the duodenal mucosa such as the ability for direct visualization during ablation (9).

Nonsurgical implants mimicking bariatric surgery have shown some level of glycemic control yet have unacceptable levels of adverse events. Promising ablative technologies are being introduced where glycemic improvements have been demonstrated and/or more clinical Investigation needs to be demonstrated. No single treatment has been demonstrated to be endoscopic and noninvasive that could target both the mucosa and the richly innervated submucosa which both play a role in resulting changes of hormonal and neural signals that are changed in bariatric surgery while at the same time not resulting in high rates of adverse events.

The present invention is directed to overcoming these and other deficiencies in the art.

### SUMMARY OF THE DISCLOSURE

The present disclosure generally relates to, inter alia, catheters, systems, and methods of using the catheters and systems for cryogenic ablation of tissue, particularly of the gastrointestinal tract.

Various aspects of the present disclosure are also addressed by the following Paragraphs 1-37 and in the noted combinations thereof, as follows:
Paragraph 1: A catheter comprising: a probe having a proximal end, a distal end, and a body portion disposed between the proximal and distal ends; and a cryogenic supply assembly housed within the probe, said cryogenic supply assembly comprising a sprayer orifice assembly and a cryogenic supply line within the sprayer orifice assembly, wherein said sprayer orifice assembly comprises a sprayer tube and a seal chamber component, said sprayer tube comprising one or more sprayer orifices for delivery of a cryogenic fluid from the cryogenic supply line to the probe, and said seal chamber component comprises a seal chamber associated with each of the one or more sprayer orifices and forming a fluidic conduit between the cryogenic supply line and the one or more sprayer orifices through which the cryogenic fluid flows to the probe, and wherein said cryogenic supply assembly is configured so that the cryogenic supply line and the sprayer tube move either axially independent of one another or axially together as one unit.
Paragraph 2: The catheter according to Paragraph 1, wherein said sprayer tube comprises a plurality of sprayer orifices.
Paragraph 3: The catheter according to Paragraph 2, wherein the plurality of sprayer orifices are arranged to include a proximal sprayer orifice, a distal sprayer orifice, and optionally one or more sprayer orifices located between the proximal and distal sprayer orifices.
Paragraph 4: The catheter according to Paragraph 2, wherein the plurality of sprayer orifices are arranged so that they are located at either the same or different circumferential positions of the sprayer tube.
Paragraph 5: The catheter according to Paragraph 1, wherein said sprayer orifice assembly further comprises an overtube disposed within the spray tube and around the cryogenic supply line.
Paragraph 6: The catheter according to Paragraph 1, wherein said sprayer orifice assembly further comprises an inner support tube disposed inside of the sprayer tube, an outer support tube disposed outside of the sprayer tube, or both an inner support tube and an outer support tube.
Paragraph 7: The catheter according to Paragraph 1, wherein said sprayer orifice assembly further comprises a restrictor component positioned over the one or more sprayer orifices and configured to cause the cryogenic fluid to exit the one or more sprayer orifices at a predetermined spray pattern, spray width, spray direction, traversing spray speed, and/or flow rate.
Paragraph 8: The catheter according to Paragraph 7, wherein the restrictor component is selected from the group consisting of a mesh, braid, a hole or plurality of holes of various shapes and sizes, and a slit or plurality of slits of various dimensions.
Paragraph 9: The catheter according to Paragraph 8, wherein the holes and slits are laser cut holes or slits of reinforced tube configured to expose a reinforcement portion of the reinforced tube.
Paragraph 10: The catheter according to Paragraph 1, wherein said sprayer orifice assembly further comprises a spray guide associated with the one or more of the sprayer orifices to control spray pattern of the cryogenic fluid as it exits the one or more sprayer orifices.
Paragraph 11: The catheter according to Paragraph 10, wherein the spray guide comprises a wall structure positioned above and encircling the one or more sprayer orifices, said wall structure extending externally from the spray tube at a desired width, wherein the spray guide is configured so that cryogenic fluid exiting from the sprayer orifice will have a spray pattern that is more constricted and/or precise as the width of the wall structure of the spray guide increases.
Paragraph 12: The catheter according to Paragraph 1 further comprising a reinforcement tube disposed around the cryogenic supply line and abutted to the sprayer tube, thereby reducing (restricting) angulation of the sprayer caused by the flow of fluid during use or reducing (restricting) the distance the sprayer moves away from the inner surface of the probe during use.
Paragraph 13: The catheter according to Paragraph 1, wherein said seal chamber component further comprises a seal stop component and an orifice seal component associated with each seal chamber.
Paragraph 14: The catheter according to Paragraph 1, further comprising a distal probe tip attached to the distal end of the probe.
Paragraph 15: The catheter according to Paragraph 1, wherein said catheter is configured for selective ablation in the mucosa and submucosa of a gastrointestinal tract of a subject.
Paragraph 16: The catheter according to Paragraph 1, wherein the treatment tissue comprises mucosal and/or submucosal tissue of the large intestine, small intestine, stomach, esophagus, rectum, and anus.
Paragraph 17: A cryogenic ablation system comprising: a catheter according to any one of Paragraphs 1-16; and a controller configured to control the functionality of the catheter for delivering a cryogenic fluid to the cryogenic catheter probe for selective ablation in mucosa and submucosa of a gastrointestinal tract of a subject.
Paragraph 18: The system according to Paragraph 17, wherein the system further comprises a shaft connected to the proximal end of the cryogenic catheter probe and/or running through all or a portion of the cryogenic catheter probe.
Paragraph 19: The system according to Paragraph 18, wherein the shaft is connected to a handle.
Paragraph 20: The system according to Paragraph 19, wherein the handle further comprises a high pressure plate.
Paragraph 21: The system according to Paragraph 20, wherein the handle further comprises a hub-cap connected to the high pressure plate.
Paragraph 22: The system according to Paragraph 17, wherein the system further comprises a pressure detection tube disposed within the cryogenic catheter probe or shaft.
Paragraph 23: The system according to Paragraph 17, wherein the cryogenic catheter probe is placed into an expanded state upon release of cryogenic fluid into the inside of the cryogenic catheter probe.
Paragraph 24: The system according to Paragraph 17, wherein the controller comprises one or more variable controller parameters used to control functional assembly.
Paragraph 25: The system according to Paragraph 24, wherein the controller is configured to perform closed-loop energy delivery to the functional assembly based on the sensor signal.
Paragraph 26: The system according to Paragraph 17, wherein the system further comprises at least one sensor constructed and arranged to produce a sensor signal.
Paragraph 27: A method of performing cryogenic ablation of mucosal tissue and/or of both mucosal tissue and submucosal tissue in the gastrointestinal tract of a subject, said method comprising: (a) providing a cryogenic ablation system according to Paragraph 17; (b) contacting the cryogenic catheter probe of the system with a target treatment region of the gastrointestinal tract of the subject; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region.
Paragraph 28: The method according to Paragraph 27, wherein the target treatment region comprises mucosal tissue and/or both mucosal and submucosal tissue of the large intestine, small intestine, stomach, esophagus, rectum, or anus of the subject.
Paragraph 29: The method according to Paragraph 28, wherein treating the target treatment region comprises performing a series of tissue ablation steps, each comprising ablation of an axial length of the large intestine, small intestine, stomach, esophagus, rectum, or anus of the subject, wherein each ablation step is optionally preceded by a tissue expansion step.
Paragraph 30: The method according to Paragraph 27, further comprises adjusting at least one variable controller parameter based on the sensor signal.
Paragraph 31: Use of a cryogenic ablation system in a method of performing cryogenic ablation of mucosal tissue and/or of both mucosal tissue and submucosal tissue in the gastrointestinal tract of a subject, wherein said method comprises: (a) providing a cryogenic ablation system according to Paragraph 17; (b) contacting the cryogenic catheter probe of the system with a target treatment region of the gastrointestinal tract of the subject; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region.
Paragraph 32: A cryogenic ablation system for use in a method of performing cryogenic ablation of mucosal tissue and/or of both mucosal tissue and submucosal tissue in the gastrointestinal tract of a subject, wherein said method comprises: (a) providing a cryogenic ablation system according to Paragraph 17; (b) contacting the cryogenic catheter probe of the system with a target treatment region of the gastrointestinal tract of the subject: and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region.
Paragraph 33: A method for performing a medical procedure in a small intestine and/or stomach of a patient in need of said medical procedure, the method comprising: (a) providing a cryogenic ablation system according to Paragraph 17; (b) contacting the cryogenic catheter probe of the system with a target treatment region of the small intestine and/or stomach of the patient; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.
Paragraph 34: The method according to Paragraph 33, wherein treating the target treatment region comprises performing a series of tissue ablation steps, each comprising ablation of an axial length of the small intestine or stomach tissue, wherein each ablation step is optionally preceded by a tissue expansion step.
Paragraph 35: The method according to Paragraph 33, further comprises adjusting at least one variable controller parameter based on the sensor signal.
Paragraph 36: Use of a cryogenic ablation system in a method for performing a medical procedure in a small intestine and/or stomach of a patient in need of said medical procedure, the method comprising: (a) providing a cryogenic ablation system according to Paragraph 17; (b) contacting the cryogenic catheter probe of the system with a target treatment region of the small intestine and/or stomach of the patient; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.
Paragraph 37: A cryogenic ablation system for use in a method for performing a medical procedure in a small intestine and/or stomach of a patient in need of said medical procedure, the method comprising: (a) providing a cryogenic ablation system according to Paragraph 17; (b) contacting the cryogenic catheter probe of the system with a target treatment region of the small intestine and/or stomach of the patient; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region, thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

In certain aspects, the present disclosure relates to a Cryogenic Ablation System for treating tissue of the esophagus, stomach, duodenum, or jejunum which comprises a catheter and controller. The catheter is delivered to the gastrointestinal tract via a delivery channel of a standard endoscope, an accessory channel mounted to the side of a standard endoscope, or inside an over a tube or delivery sheath. All three methods allow the user to position the endoscope on the distal end of the catheter facilitating visualization through the distal end of the catheter.

In certain aspects, the catheter encompasses a probe, a shaft, a handle, a high-pressure catheter connector, and a distal tip. The probe increases and decreases in diameter to conform within the gastrointestinal tract's anatomical passageways (e.g., esophagus, stomach, duodenum, or jejunum). When the probe is fully inflated, it conforms within the passageway of the treatment area, securing its position at the desired treatment location while using the endoscope's visualization.

In certain aspects, the catheter comprises: a probe having a proximal end, a distal end, and a body portion disposed between the proximal and distal ends; and a sprayer orifice assembly housed within the probe. The sprayer orifice assembly is comprised of the following: a cryogenic supply line for delivery of cryogenic fluid, a cryogenic supply line hole which allows cryogen to pass into a seal chamber inside of the sprayer tube which contains one or more sprayer orifices.

The probe's distal end is mounted to a support tube which is connected to an over tube, which runs through the distal tip, and the proximal end of the probe is mounted to the shaft. The cryogenic supply line may move axially and independently relative to the sprayer tube and the sprayer tube may move axially and independently relative to the cryogenic supply line. This axial movement allows for selective fluid delivery to one or more sprayer orifices. Further, once a sprayer orifice is selected, the cryogenic supply line and sprayer tube may travel axial in unison delivering cryogenic fluid in an axial pattern to the inside of the probe.

In certain aspects, the sprayer orifice may be covered with a mesh type structure, or a mesh type structure may be incorporated into the wall of the sprayer tube and sprayer orifice facilitating the distribution of the cryogenic fluid to the inside of the probe.

In certain aspects, the controller encompasses a control system, a cryogenic fluid delivery system, a cryogenic supply system, and the controller connector. The control system contains hardware, software with associated algorithms, and a user interface, defining treatment parameters. The cryogenic fluid delivery system encompasses one or more control valves, one or more diverter valves, one or more motors, and one or more exhaust ports, which together with the control system automatically controls and directs the flow of cryogenic fluid at a predetermined time, a predetermined flow rate, to a selected cryogenic delivery channel(s), and cryogenic delivery channel(s) position, direction, translation speed. The cryogenic supply system encompasses one or more cryogen cartridges that supply the cryogenic fluid for treatment. Once the high-pressure catheter connector is coupled to the controller connector, one or more cryogenic fluid delivery channels are fluidly coupled to the one or more canisters of cryogenic fluid.

In certain aspects, upon release from the controller seal the cryogenic fluid is directed through the cryogenic supply line, through the cryogenic supply line hole, and into the seal chamber, flowing outwardly through one or more of the sprayer orifices toward the probe inner surface, causing the probe diameter to increase, and come into intimate contact with the tissue of the gastrointestinal tract. The cryogenic ablation system is now ready for treatment. Based on the controller system, the present invention which accurately controls probe inflation rate, allowing the probe to increase diameter for intimate contact with gastrointestinal tissue, probe inflation pressure, and probe temperature.

In certain aspects, upon delivery of cryogenic fluid during treatment, cryogen makes contact with the inside of the probe wall causing cells in contact with the probe wall (the inner layer of the mucosa) to undergo rapid freezing rates, whereas cells in the periphery of the ablation interface (submucosa and deeper layers of the gastrointestinal tract to undergo moderate to lower freezing rates (10).

In certain aspects, cryoablation via the application of liquid cryogen through the introduction into an expandable probe in intimate contact with tissue of the gastrointestinal tract (mucosa) specifically of the small intestine leads to regenerated small intestine tissue. The regenerative effect is from the mucosa and into the submucosa where critical microvasculature in the submucosa is preserved (11). Temperature decrease to the submucosa facilitates modulation of the nerves of the submucosa (partial or reversible ablation, blocking, stimulation) while leaving critical microvascular structures intact which are necessary for regeneration. Further, the rapid freezing rates delivered to mucosal tissue modifies integrity and function of the intestinal barrier (mucosal epithelium) wherein the target mucosa intercellular spaces (ICS) decrease, and MI (mucosal impedance) increase thereby decreasing the permeability in mucosa ablated (12, 13).

In contrast to radiofrequency ablation, laser ablation, heated fluid ablation, and potentially other heat-based ablation techniques, cryoablation results in a robust inflammatory response, particularly in the submucosa. The inflammatory response creates the potential to stimulate additional responses particularly immunologic responses in cryoablation of tumors (14). Commonly employed ablation therapies in the clinical setting are radiofrequency ablation (RFA), microwave ablation, high-intensity focused ultrasound, and cryoablation. All these treatments operate on the principle of hyperthermia except for cryoablation, which is a hypothermic modality that induces tissue damage by a freeze-thaw process. Of all the ablation techniques, cryoablation demonstrated the highest potential to elicit a post-ablative immunogenic response (15). Finally, peptide bonds are not disrupted in 20) the process of cryoablation so cold denaturation of proteins can be reversible with warming and rehydration (16).

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, if provided, like reference numerals contained in the drawings are meant to identify similar or identical elements.

FIG. 1 is a perspective view of the cryogenic ablation system and portions of the catheter and controller.

FIG. 2 is illustrative of the catheter exiting the endoscope within the duodenum positioned for treatment.

FIG. 3 is an internal view of the controller displaying the load cap(s) and cartridge containers.

FIG. 4 is a perspective view of the catheter.

FIG. 5 is a perspective view of the cryogenic supply assembly overview.

FIG. 6 is a perspective view of the sprayer orifice assembly.

FIG. 7A is a perspective view of the cryogenic supply line solid tube.

FIG. 7B is a perspective view of the cryogenic supply line polyimide braided.

FIG. 8 is a side view of the cryogenic supply line polyimide variable braid.

FIG. 9A is a perspective view of the overtube solid tube.

FIG. 9B is a perspective view of the overtube polyimide braid.

FIG. 10 is a side view of the overtube polyimide variable braid.

FIG. 11 is a cross-sectional view of the catheter shaft tri-lumen extrusion.

FIG. 12 is a cross-sectional view of the catheter shaft tri-lumen extrusion with support member.

FIG. 13 is a side view of the sprayer orifice assembly with mesh incorporated within the sprayer tube.

FIG. 14A is a perspective view of the sprayer tube opening as mesh is placed.

FIG. 14B is a perspective view of the sprayer tube opening with mesh secured.

FIG. 15 is a side view of the sprayer orifice assembly with mesh wrapped around sprayer tube.

FIG. 16 is a side view of the sprayer orifice assembly with a hole pattern incorporated within sprayer tube.

FIG. 17 is various sprayer orifice pattern designs.

FIG. 18 is a side view of the spray tube with two sprayer orifices.

FIG. 19 is a section view of the sprayer orifice assembly with sprayer guide shown.

FIG. 20A is a side view of the sprayer orifice assembly demonstrating a spray pattern without a spray guide.

FIG. 20B is a side view of the sprayer orifice assembly demonstrating a spray pattern with a spray guide.

FIG. 21 is side view of a sprayer orifice assembly.

FIG. 22A is a top view of the sprayer orifice assembly where the distal sprayer is selected.

FIG. 22B is a top view of the sprayer orifice assembly where the proximal sprayer is selected.

FIG. 23A is a top view of the sprayer orifice assembly where the distal sprayer is selected.

FIG. 23B is a top view of the sprayer orifice assembly where the proximal sprayer is selected.

FIG. 24 is a side view of the probe in the load position.

FIG. 25 is a side view of the catheter connector handle in the load position.

FIG. 26 is a side view of the probe inflating while in the sprayer orifice assembly is in the distal home position.

FIG. 27 is a side view of the catheter connector handle in the distal home position for the distal sprayer orifice.

FIG. 28 is a side view of the probe in the distal position for distal sprayer orifice, the flow of cryogen fluid is initiated.

FIG. 29 is a side view of the catheter connector handle in the proximal position for distal sprayer orifice.

FIG. 30 is a side view of the probe in the proximal position for distal sprayer orifice, the flow of cryogen fluid is initiated.

FIG. 31 is a side view of the catheter connector handle in the distal home position for the proximal sprayer orifice.

FIG. 32 is a side view of the probe in the distal position for proximal sprayer orifice, the flow of cryogen fluid is initiated.

FIG. 33 is a side view of the catheter connector handle in the proximal position for distal sprayer orifice.

FIG. 34 is a side view of the probe in the proximal position for proximal sprayer orifice, the flow of cryogen fluid is initiated.

It should be understood that the drawings are not necessarily to scale. In certain instances, details that are not necessary for an understanding of the invention or that render other details difficult to perceive may have been omitted. It should be understood, of course, that the invention is not necessarily limited to the particular embodiments illustrated herein.

### DETAILED DESCRIPTION OF THE DISCLOSURE

The present disclosure provides medical devices (e.g., catheters), systems, and methods for use of the devices and systems for selectively ablating of the mucosa and submucosa in the gastrointestinal tract, including, inter alia, for the treatment of Type 2 Diabetes, obesity, and other metabolic conditions.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises.” “has,” “includes” or “contains” one or more elements possesses those one or more elements but is not limited to possessing only those one or more elements. Likewise, an element of a system, device, or apparatus that “comprises,” “has,” “includes” or “contains” one or more features possesses those one or more features but is not limited to possessing only those one or more features. Likewise, an element of a system, device, or apparatus that “comprises.” “has,” “includes,” or “contains” one or more features possesses those one or more elements but is not limited to keeping only those one or more attributes.

The terms “proximal” and “distal” are used herein regarding a clinician manipulating the controller portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those of ordinary skill in the art will recognize various equivalent variations on the description that follows. Unless otherwise stated, in this application, specified relationships, such as parallel to, aligned with, or in the same plane as, mean that the specified relationships are within limitations of manufacturing processes and manufacturing variations. When components are described as being coupled, connected, being in contact, or contacting one another, they need not be physically directly touching one another unless specifically described as such. Like elements in various embodiments are commonly referred to with like reference numerals.

Table A is a listing of the reference numbers for the various elements or items shown in the as-filed FIGURES. Where applicable, the reference numbers of Table A can be used interchangeably with the Figures and text of the element/item in the present disclosure.

TABLE A

| Ref. No. | Element/Item | FIGS. |
|---|---|---|
| 1 | Agil's Cryoablation | 2, 7B, 8, 9B, 10 |
| 2 | Axial Reinforcement | 7B, 8, 9B, 10 |
| 3 | Braiding | 7B, 9B |
| 4 | Cartridge Container | 3 |
| 5 | Catheter | 2 |
| 6 | Catheter Connector Handle | 4, 5, 25, 27, 29, 31, 33 |
| 7 | Catheter Locking Motor | 3 |
| 8 | Catheter Shaft | 4, 24, 25, 26, 27, 28, 29, 30, 31, |
| | | 32, 33, 34 |
| 9 | Catheter Shaft Tri-Lumen Extrusion | 11 |
| 10 | Catheter Shaft Tri-Lumen Extrusion with | 12 |
| | Support Member | |
| 11 | Catheter Tip | 4, 24, 30, 32, 34, 26, 28 |
| 12 | Controller | 1 |
| 13 | Controller Connector | 1, 3 |
| 14 | Cryogenic Fluid | 13, 15, 16, 20A, 20B, 22A, 22B, |
| | | 23A, 23B |
| 15 | Cryogenic Fluid Deliver System | 3 |
| 16 | Cryogenic Spray | 28 |
| 17 | Cryogenic Supply Assembly Lumen | 11, 12 |
| 18 | Cryogenic Supply Assembly Overview | 5 |
| 19 | Cryogenic Supply Block | 3 |
| 20 | Cryogenic Supply Line | 7A, 7B, 8, 13, 15, 16, 19, 21, |
| | | 22A, 22B, 23A, 23B |
| 21 | Cryogenic Supply Line Hole Double Wall | 22A, 22B |
| 22 | Cryogenic Supply Line Hole Double Wall | 22B |
| 23 | Cryogenic Supply Line Sealed | 22A, 22B |
| 24 | Cryogenic Supply System | 3 |
| 25 | Cryogenic Supply Assembly Hole Pattern | 16 |
| | Incorporated within Sprayer Tube | |
| 26 | Cryogenic Supply Assembly Mesh | 13 |
| | Incorporated within Sprayer Tube | |
| 27 | Cryogenic Supply Assembly Mesh | 15 |
| | Wrapped around Sprayer Tube | |
| 28 | Delivery Channel Flow Valve | 3 |
| 29 | Distal Home Position | 27, 28, 31, 32 |
| 30 | Distal Orifice Selected | 22A |
| 31 | Distal Sprayer Orifice | 6, 18, 21, 22A, 2B, 23A, 23B, 24, |
| | | 26, 28, 30, 32, 34 |
| 32 | Duodenum | 2 |
| 33 | Exhaust Port | 4, 25, 27, 29, 31, 33 |
| 34 | Exhaust Chamber | 3 |
| 35 | Exhaust Lumen | 11, 12 |
| 36 | Exhaust Valve | 3 |
| 37 | Exterior Wall | 11, 12 |
| 38 | Flow/Pressure PCB | 3 |
| 39 | Gas Inflation | 26 |
| 40 | Handle | 1 |
| 41 | High Pressure Connector | 3 |
| 42 | High Pressure Port | 25, 27, 29, 31 |
| 43 | Inflating/Distal Home Position | 26 |
| 44 | Less Braiding, Stiff Section | 8, 10 |
| 45 | Load Position | 25 |
| 46 | Main Flow Valve | 3 |
| 47 | Mesh | 13, 14A, 15, 19 |
| 48 | Mesh secured to opening creating Sprayer | 14B |
| | Orifice | |
| 49 | More Braiding, Flexible Section | 8, 10 |
| 50 | Opening | 14A |
| 51 | Orifice Seal | 19, 21, 22A, 22B, 23A, 23B |
| 52 | Outer Support | 19, 21 |
| 53 | Overtube | 5, 6, 9A, 9B, 10, 13, 15, 16 |
| 54 | Polyimide Tube | 7B, 8, 9B, 10 |
| 55 | Probe | 4, 20A, 20B, 26, 28, 30, 32, 34 |
| 56 | Probe Pressure Lumen | 11, 12 |
| 57 | Probe Pressure Port | 4, 25, 27, 29, 31, 33 |
| 58 | Probe Proximal Orifice | 34 |
| 59 | Proximal Orifice Selected | 22B |
| 60 | Proximal Sprayer Orifice | 6, 18, 19, 21, 22A, 22B, 23A, |
| | | 23B, 24, 26, 28, 30, 32, 34 |
| 61 | Seal Chamber | 19, 21 |
| 62 | Seal Stop | 19, 21, 22A, 22B, 23A, 23B |
| 63 | Solid Tube | 7A |
| 64 | Spray Guide | 19, 20B |
| 65 | Spray Pattern | 20A, 20B |
| 66 | Sprayer Orifice | 13, 15, 16 |
| 67 | Sprayer Orifice Assembly | 5, 6, 13, 15, 16, 19, 20A, 20B, 21, |
| | | 22A, 22B, 23A, 23B, 24, 28, 30, |
| | | 32, 34 |
| 68 | Sprayer Orifice Holes and Patterns | 17 |
| 69 | Sprayer Selector | 25, 27, 29, 31 |
| 70 | Sprayer Translation Assembly Hub | 4, 25, 27, 29, 31, 33 |
| 71 | Sprayer Tube | 6, 13, 14A, 14B, 15, 16, 18, 19, |
| | | 21, 22A, 22B, 23A, 23B |
| 72 | Sprayer Tube Mesh Wrapped around | 14B |
| | Sprayer Tube | |
| 73 | Sprayer Tube Opening Mesh Placement | 14A |
| 74 | Sprayer Tube with two Sprayer Orifices | 18 |
| 75 | Stomach | 2 |
| 76 | Support Member | 12 |
| 77 | Support Tube | 5, 6 13, 15, 16 |
| 78 | Traversed to Proxima Position | 29, 30, 33, 34 |
| 79 | Treatment Motor | 3 |

In one aspect, the present disclosure provides a catheter for selective ablation in the mucosa and submucosa of the gastrointestinal tract, said catheter comprising elements as disclosed and/or contemplated herein.

In another aspect, the present disclosure provides a catheter that includes: a probe having a proximal end, a distal end, and a body portion disposed between the proximal and distal ends; and a cryogenic supply assembly housed within the probe. The cryogenic supply assembly includes a sprayer orifice assembly and a cryogenic supply line within the sprayer orifice assembly, where the sprayer orifice assembly includes a sprayer tube and a seal chamber component, the sprayer tube includes one or more sprayer orifices for delivery of a cryogenic fluid from the cryogenic supply line to the probe, and the seal chamber component includes a seal chamber associated with each of the one or more sprayer orifices and forming a fluidic conduit between the cryogenic supply line and the one or more sprayer orifices through which the cryogenic fluid flows to the probe, and where the cryogenic supply assembly is configured so that the cryogenic supply line and the sprayer tube move either axially independent of one another or axially together as one unit. In use according to the present disclosure, the axial movement of the sprayer orifice assembly in relation to the cryogenic supply line can be executed with or without the delivery of the cryogenic fluid from the one or more sprayer orifices to the probe. In certain embodiments, the cryogenic supply assembly is configured so that the cryogenic supply line and the sprayer tube move either axially independent of one another or axially together as one unit, without requiring any rotational movement relative to each other. Furthermore, in certain embodiments, the catheter of the present disclosure is configured for selective delivery of the cryogenic fluid to one seal chamber individually, which eliminates the need for rotation of the sprayer tube in relation to the cryogenic supply line or vice versa.

In certain embodiments, the sprayer tube includes one sprayer orifice. In certain other embodiments, the sprayer tube includes a plurality of sprayer orifices. The plurality of sprayer orifices can be arranged to include a proximal sprayer orifice, a distal sprayer orifice, and optionally one or more sprayer orifices located between the proximal and distal sprayer orifices. In use according to the present disclosure, the different sprayer orifices can used to independently of one another, including one at a time, so that the cryogenic fluid exits only one sprayer orifice at a given time.

In certain embodiments, the plurality of sprayer orifices are arranged so that they are located at either the same or different circumferential positions of the sprayer tube. For example, in certain embodiments, the sprayer orifices can be located at different radial dimensions (1° to 360°) around the sprayer tube and at different axial positions along the sprayer tube. In certain embodiments, there can be one or more sprayer orifices per seal chamber.

In certain embodiments, the sprayer orifice assembly further includes an overtube disposed within the spray tube and around the cryogenic supply line.

In certain embodiments, the sprayer orifice assembly further includes an inner support tube disposed inside of the sprayer tube, an outer support tube disposed outside of the sprayer tube, or both an inner support tube and an outer support tube. As provided herein, both the inner support tube and the outer support tube function to support the one or more sprayer orifices, with the inner support tube positioned inside the sprayer tube and the outer support tube positioned outside of the sprayer tube. In addition to this support function, the inner support tube can also function for the secondary purpose of reducing the internal volume of the seal chamber. This reduction is useful to prevent or minimize the cryogenic liquid from undergoing a phase change, ensuring it remains liquid.

In certain embodiments, the sprayer orifice assembly further includes a restrictor component positioned over the one or more sprayer orifices and configured to cause the cryogenic fluid to exit the one or more sprayer orifices at a predetermined spray pattern, spray width, spray direction, traversing spray speed, and/or flow rate. In certain embodiments, the restrictor component can include a mesh, braid, a hole or plurality of holes of various shapes and sizes, a slit or plurality of slits of various dimensions, and the like. In certain embodiments, the holes and slits are laser cut holes or slits of reinforced tube configured to expose a reinforcement portion of the reinforced tube. In certain embodiments, the restrictor component can be over, below, or within the mesh, braid, etc., and can be part of the sprayer tube and exposed.

In certain embodiments, the sprayer orifice assembly further includes a spray guide associated with the one or more of the sprayer orifices to control spray pattern of the cryogenic fluid as it exits the one or more sprayer orifices.

In certain embodiments, the spray guide includes a wall structure positioned above and encircling the one or more sprayer orifices, with the wall structure extending externally from the spray tube at a desired width, where the spray guide is configured so that cryogenic fluid exiting from the sprayer orifice will have a spray pattern that is more constricted and/or precise as the width of the wall structure of the spray guide increases.

In accordance with the present disclosure, the sprayer guide and the restrictor component can be used to yield a desired spray pattern of the cryogenic fluid from the sprayer orifice, ranging from a shotgun type of spray pattern to a more precise spray pattern that focuses on a specific target point or area.

In certain embodiments, the catheter of the present disclosure further includes a reinforcement tube disposed around the cryogenic supply line and abutted to the sprayer tube, thereby reducing (restricting) angulation of the sprayer caused by the flow of fluid during use or reducing (restricting) the distance the sprayer moves away from the inner surface of the probe during use. In certain embodiments, the reinforcement tube is configured to have various lengths suitable for use with a catheter shaft. For example, in certain embodiments, the reinforcement tube is configured to terminate within at least one inch (1 inch) within the catheter shaft.

In certain embodiments, the seal chamber component further includes a seal stop component and an orifice seal component associated with each seal chamber.

In certain embodiments, the catheter of the present disclosure further includes a distal probe tip attached to the distal end of the probe.

In certain embodiments, the catheter of the present disclosure is configured for selective ablation in the mucosa and submucosa of a gastrointestinal tract of a subject.

In certain embodiments, the catheter of the present disclosure is effective for use in the treatment of various tissues of a subject, including, without limitation, mucosal and/or submucosal tissue of the large intestine, small intestine, stomach, esophagus, rectum, and anus.

In another aspect, the present disclosure provides a cryogenic ablation system including: a catheter according to the present disclosure; and a controller configured to control the functionality of the catheter for delivering a cryogenic fluid to the cryogenic catheter probe for selective ablation in mucosa and submucosa of a gastrointestinal tract of a subject.

In certain embodiments, this system further includes a shaft connected to the proximal end of the cryogenic catheter probe and/or running through all or a portion of the cryogenic catheter probe. In certain embodiments, the shaft is connected to a handle. In certain embodiments, the handle further includes a high pressure plate. In certain embodiments, the handle further includes a hub-cap connected to the high pressure plate.

In certain embodiments, this system further includes a pressure detection tube disposed within the cryogenic catheter probe or shaft. In certain embodiments, the cryogenic catheter probe is placed into an expanded state upon release of cryogenic fluid into the inside of the cryogenic catheter probe. In certain embodiments, the controller includes one or more variable controller parameters used to control functional assembly. In certain embodiments, the controller is configured to perform closed-loop energy delivery to the functional assembly based on the sensor signal.

In certain embodiments, this system further includes at least one sensor constructed and arranged to produce a sensor signal.

In another aspect, the present disclosure provides a method of performing cryogenic ablation of mucosal tissue and/or of both mucosal tissue and submucosal tissue in the gastrointestinal tract of a subject. This method involves: (a) providing a cryogenic ablation system according to the present disclosure: (b) contacting the cryogenic catheter probe of the system with a target treatment region of the gastrointestinal tract of the subject; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region.

In certain embodiments of this method, the target treatment region includes mucosal tissue and/or both mucosal and submucosal tissue of the large intestine, small intestine, stomach, esophagus, rectum, or anus of the subject.

In certain embodiments of this method, the treating of the target treatment region includes performing a series of tissue ablation steps, each including ablation of an axial length of the large intestine, small intestine, stomach, esophagus, rectum, or anus of the subject, where each ablation step is optionally preceded by a tissue expansion step.

In certain embodiments, this method further involves adjusting at least one variable controller parameter based on the sensor signal.

In another aspect, the present disclosure involves the use of a cryogenic ablation system in a method of performing cryogenic ablation of mucosal tissue and/or of both mucosal tissue and submucosal tissue in the gastrointestinal tract of a subject, where the method involves: (a) providing a cryogenic ablation system according to the present disclosure: (b) contacting the cryogenic catheter probe of the system with a target treatment region of the gastrointestinal tract of the subject; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region.

In another aspect, the present disclosure provides a cryogenic ablation system for use in a method of performing cryogenic ablation of mucosal tissue and/or of both mucosal tissue and submucosal tissue in the gastrointestinal tract of a subject, where the method involves: (a) providing a cryogenic ablation system according to the present disclosure: (b) contacting the cryogenic catheter probe of the system with a target treatment region of the gastrointestinal tract of the subject; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region.

In another aspect, the present disclosure provides a method for performing a medical procedure in a small intestine and/or stomach of a patient in need of the medical procedure, the method involving: (a) providing a cryogenic ablation system according to the present disclosure; (b) contacting the cryogenic catheter probe of the system with a target treatment region of the small intestine and/or stomach of the patient; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region, thereby performing a medical procedure to treat a condition of the patient that can include, without limitation, Type 1 diabetes, Type 2 diabetes, non-alcoholic fatty liver disease (NAFLD), non-alcoholic steatohepatitis (NASH), and/or obesity.

In certain embodiments of this method, treating the target treatment region involves performing a series of tissue ablation steps, each including ablation of an axial length of the small intestine or stomach tissue, where each ablation step is optionally preceded by a tissue expansion step.

In certain embodiments, this method further involves adjusting at least one variable controller parameter based on the sensor signal.

In another aspect, the present disclosure involves the use of a cryogenic ablation system of the present disclosure in a method for performing a medical procedure in a small intestine and/or stomach of a patient in need of said medical procedure, where the method involves: (a) providing a cryogenic ablation system according to the present disclosure; (b) contacting the cryogenic catheter probe of the system with a target treatment region of the small intestine and/or stomach of the patient; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region, thereby performing a medical procedure to treat a condition of the patient that can include, without limitation, Type 1 diabetes, Type 2 diabetes, non-alcoholic fatty liver disease (NAFLD), non-alcoholic steatohepatitis (NASH), and obesity.

In another aspect, the present disclosure provides a cryogenic ablation system for use in a method for performing a medical procedure in a small intestine and/or stomach of a patient in need of the medical procedure, where the method involves: (a) providing a cryogenic ablation system according to the present disclosure; (b) contacting the cryogenic catheter probe of the system with a target treatment region of the small intestine and/or stomach of the patient; and (c) releasing a cryogenic fluid from the one or more sprayer orifices to treat the target treatment region by cryogenically ablating at least a portion the mucosal tissue or ablating at least a portion of both the mucosal and submucosal tissue of the target treatment region, thereby performing a medical procedure to treat a condition of the patient that can include, without limitation, Type 1 diabetes, Type 2 diabetes, non-alcoholic fatty liver disease (NAFLD), non-alcoholic steatohepatitis (NASH), and obesity.

In one embodiment, the catheter comprises: a probe having a proximal end, a distal end, and a body portion disposed between the proximal and distal ends; and a sprayer orifice assembly housed within the probe, said sprayer orifice assembly comprising a cryogenic supply line for delivery of cryogenic fluid through a cryogenic supply line hole to a seal chamber.

In certain embodiments, the cryogenic supply line is within a sprayer tube where the space between the cryogenic supply line and the sprayer tube may consist of two or more seal stops, and two or more orifice seals, creating individual seal chambers.

In certain embodiments, the seal chambers allow for selective delivery of cryogenic fluid to one or more sprayer orifices.

In certain embodiments, the cryogenic supply line may be reinforced with an overtube which provides axial support to the cryogenic supply line.

In certain embodiments, the sprayer tube has from 1 to 8 sprayer orifices.

In certain embodiments, the sprayer orifices are distributed around the circumference of the sprayer tube and at various axial positions along the sprayer tube.

In certain embodiments, the arc length of each sprayer orifice can be designed between 5 and 360 degrees.

In certain embodiments, the sprayer orifice may be a slit.

In certain embodiments, the sprayer orifice may be pattern of shapes creating the surface area that makes up each sprayer orifice.

In certain embodiments, the sprayer orifice may be covered or wrapped with mesh.

In certain embodiments, mesh may be incorporated into the wall of the sprayer tube and within the sprayer orifice.

In certain embodiments, the mesh can be welded, woven, or sintered with various mesh wire diameters where the mesh wires are woven at various patterns creating evenly spaced openings where the openings sizes can vary from one mesh to another mesh.

In certain embodiments, the cryogenic supply line may move axially and independently relative to the sprayer tube.

In certain embodiments, the sprayer tube may move axially and independently relative to the cryogenic supply line.

In certain embodiments, the cryogenic supply line, overtube, and sprayer tube may move axially as a unit.

In certain embodiments, the axial positioning and movement of the cryogenic supply line and/or the sprayer tube may create a seal chamber in direct fluid communication with the cryogenic supply line and one or more sprayer orifices.

In certain embodiments, the user can select a specific axial position of cryogenic supply line or the sprayer tube where one or more specific sprayer orifices are selected to delivery cryogenic fluid to a specific area within the probe.

In certain embodiments, the catheter further comprises a shaft connected to the proximal end of the probe and/or running through all or a portion of the probe.

In certain embodiments, the seal chamber which is comprised of seal stops and orifice seals may be incorporated with the sprayer tube and move axially with the sprayer tube.

In certain embodiments, the seal chamber which is comprised of seal stops and orifice seals may be incorporated with the sprayer tube and move axially with the sprayer tube.

In certain embodiments, the shaft is connected to a catheter connector handle.

In certain embodiments, the catheter connector handle further comprises an exhaust port, sprayer translation assembly hub, and a probe pressure port.

In certain embodiments, the catheter further comprises a pressure detection lumen disposed within the probe or shaft.

In another aspect, the present disclosure provides a system for selective ablation in the mucosa and submucosa of the gastrointestinal tract, said system comprising components as contemplated and/or described herein.

In embodiment, the system comprises a catheter as disclosed and/or contemplated herein; and a controller as disclosed and/or contemplated herein.

In another aspect, the present disclosure provides a method for performing a medical procedure in an intestine of a patient, the method comprising: (a) providing a catheter as disclosed and/or contemplated herein for insertion into the intestine or a system comprising the catheter and a controller, said catheter comprising: (i) proximal and distal portions; (ii) a probe mounted to the distal portion; and (iii) one or more sprayer orifices delivering cryogen to the inside of the probe; (b) introducing the catheter into the patient; and (c) treating target tissue with the probe in contact with the target tissue, wherein the target tissue comprises mucosal (and/or submucosal) tissue of the small intestine and treatment comprises ablating at least a portion of the mucosal and submucosal tissue of the small intestine, and wherein the medical procedure is configured to treat at least one of type 2 diabetes, non-alcoholic fatty liver disease (NAFLD) or non-alcoholic steatohepatitis (NASH). Suitable aspects of the present disclosure can be used in accordance with the disclosure of U.S. Pat. No. 10,610,663.

In another aspect, the present disclosure provides a method for ablating (regenerating) the mucosa of the small intestine and alter, stimulate, or reduce neural activity in the submucosa of the small intestine of a subject, the method comprising: (a) providing a catheter for insertion into the intestine or a system comprising the catheter and a controller, said catheter comprising: (i) proximal and distal portions: (ii) a probe mounted to the distal portion; and (iii) one or more cryogenic fluid delivery channels delivering cryogen to the wall of the small intestine, at a selected power density (W/cm²) and a selected energy density (J/cm²), wherein the cryogenic fluid delivery channel can delivery cryogen that can elevate (or lower) tissue temperature from −25° C. to −190C; and (b) lowering a temperature of the target area using the cryogenic fluid delivery channels of the catheter, thereby ablating the mucosa of the intestine and delivers therapeutic energy to the submucosa to at least partially alter, stimulate, or reduce the neural activity or within the submucosal layer while maintaining functional activity of other layers of the surrounding target area. Suitable aspects of the present disclosure can be used in accordance with the disclosure of U.S. Pat. No. 10,537,387.

In certain embodiments, the medical procedure is further configured to treat a disease or disorder selected from the group consisting of: Type 2 diabetes: Type 1 diabetes: “Double diabetes”; gestational diabetes: hyperglycemia: pre-diabetes: impaired glucose tolerance: insulin resistance; and combinations thereof. Suitable aspects of the present disclosure can be used in accordance with the disclosure of U.S. Pat. No. 10,610,663.

In certain embodiments, treating target tissue modifies at least one of (1) nutrient absorption by the target tissue, (2) hormonal signaling from the target tissue, (3) secretions of the target tissue. Suitable aspects of the present disclosure can be used in accordance with the disclosure of U.S. Pat. No. 10,610,663.

In certain embodiments, treating target tissue modifies integrity and function of the intestinal barrier (mucosal epithelium) wherein the target mucosa intercellular spaces (ICS) decrease, and MI (mucosal impedance) increase thereby decreasing the permeability in mucosa ablated.

In certain embodiments, the sensory nerves comprise at least one nerve that is activated by food passing through the duodenum; and/or wherein the sensory nerves comprise at least one nerve that transmits signals from at least one of mechano-sensors or chemoreceptors located within the duodenal wall. Suitable aspects of the present disclosure can be used in accordance with the disclosure of U.S. Pat. No. 10,537,387.

In certain embodiments, cryogenic fluid delivery channel(s) may be independently controlled and turned to an on or off position.

In certain embodiments, the sprayer orifice assembly, comprising a sprayer tube and one or more sprayer orifice(s), cryogenic supply line, a seal chamber, and a cryogenic supply line hole the cryogenic supply line has a distal and proximal end: wherein said delivery sprayer orifice is connected to the distal end of the cryogenic supply line via the cryogenic supply hole and seal chamber; and wherein the sprayer orifice constricts flow of cryogenic fluid minimizing liquid to gas expansion in cryogenic fluid delivery channel.

In certain embodiments, the cryogenic supply line is fluidly connected to a delivery channel flow valve and a reservoir of cryogenic fluid whereby cryogenic fluid delivery channel can be controlled by actuation of each delivery channel flow valve.

In certain embodiments, each sprayer orifice allows for partial restriction of cryogenic fluid and a delivery channel flow valve controls release of cryogenic fluid from the cryogenic supply block into each cryogenic fluid delivery channel.

In certain embodiments, the controller independently controls deliver of cryogenic fluid to each delivery channel via control valves at the proximal end of each delivery channel, and/or wherein a reservoir system allows for large ablation areas up to 10 cm of tissue in a partial-circumferential or full-circumferential ablations.

In certain embodiments, the probe will be placed into an expanded state upon release of cryogenic fluid into the inside of the probe.

In certain embodiments, the system further comprises a controller operably attached to the functional assembly, and wherein the controller comprises one or more variable controller parameters used to control the functional assembly.

In certain embodiments, the system further comprises at least one sensor constructed and arranged to produce a sensor signal, and wherein the method further comprises adjusting at least one variable controller parameter based on the sensor signal.

In certain embodiments, the controller is configured to perform closed-loop energy delivery to the functional assembly based on the sensor signal.

In certain embodiments, treating target tissue comprises a series of tissue ablation steps, each comprising ablation of an axial length of intestinal tissue, wherein each ablation step is preceded by a tissue expansion step.

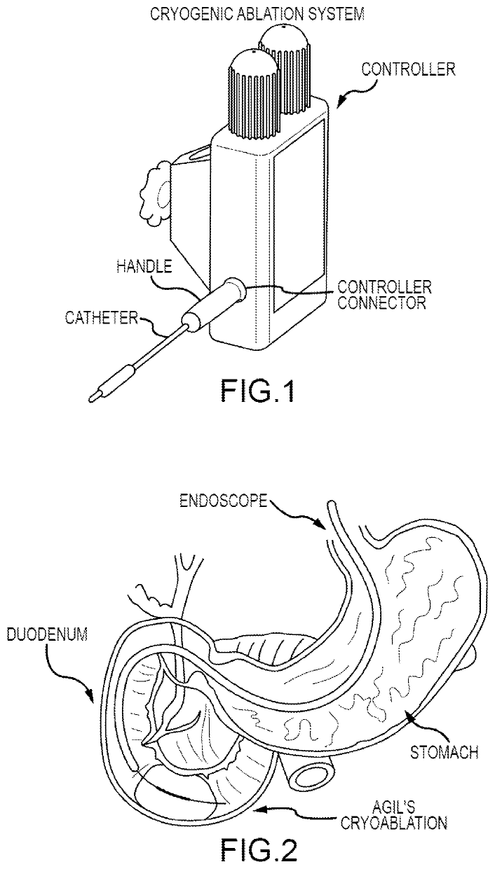

Referring now to FIG. 1, a schematic view of the Cryogenic Ablation System, consisting of a controller and a catheter. The catheter shown in FIG. 1 includes a catheter constructed according to the present invention's teachings and has a distal end and a proximal end. The proximal end of the catheter carries a connecting member (catheter high-pressure connector), through which the catheter is securely received into the controller. The catheter may be for single use, whereas the controller is reusable. The catheter is shown in the attached configuration with the controller.

Referring now to FIG. 2, an illustration of the embodiment of a Cryogenic Ablation System for performing a medical procedure on a patient. The Cryogenic Ablation System comprises a catheter and a controller. In this illustration, an embodiment of the catheter includes a probe which is a component of the catheter and is positioned and expanded for intimate contact with the targeted tissue as illustrated. The catheter is used in conjunction with an endoscope as illustrated. The distal end of such an endoscope in FIG. 2, has an imaging camera lens and illuminating light. The image is picked up by the lens is transferred via fiber optics to a monitoring camera, which sends TV signals via a cable to a conventional monitor, where the procedure can be visualized. By virtue of this visualization, the physician can perform cryoablation in the gastrointestinal tract. The catheter is configured to pass through the working channel of an endoscope. The probe is positioned on a distal portion of the catheter and is configured to be inflated by introducing cryogenic fluid into the catheter where the diameter of the probe may be increased or decreased to facilitate the 25 introduction, removal, or positioning of the catheter and treatment using the catheter within the anatomical passageways.

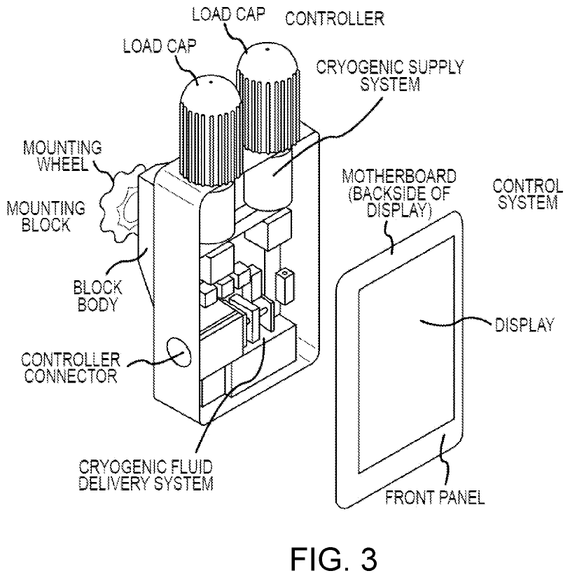

Referring to FIG. 3 is an embodiment of the controller with the front cover removed for better visualization of the internal components. The controller's cryogenic supply system may include one or more load caps, one or more cartridges (residing within the load caps), one or more of the cartridge containers, and a cryogenic supply block that connects the cryogenic supply system to the cryogenic fluid delivery system. The cryogenic fluid delivery system consists of one or more main flow valves controlling the flow of cryogenic fluid from the cryogenic supply system into one or more delivery channel flow valve(s) which controls the flow of cryogenic fluid into one or more cryogenic fluid delivery channel(s). The control system controls the main flow valve(s) and delivery channel flow valve(s). The control system controls the cryogenic liquid delivery system to automatically direct the cryogenic fluid flow at a predetermined start and stop time by using an electronic or mechanical timer. The control system controls the linear traversing rate that is either distal or proximal by means of the treatment motor with an optical sensor or magnetic reader that translates the channels within the probe at a precise rate and location The controller connector aligns the appropriate ports from the high-pressure catheter connector, so the cryogenic supply system is fluidly coupled to the cryogenic fluid delivery channel(s). The cryogenic fluid delivery system encompasses the catheter locking motor, which keeps the catheter securely in place and connected with the controller at the catheter connector manifold. Within cryogenic fluid delivery system can be configured with liquid flow port, exhaust, and probe pressure port. Measurements from these ports may be inputs to a control algorithm implemented on the cryogenic liquid delivery system. The operation of the controller may be regulated or adjusted based on sensor feedback. In some embodiments, it may be desirable for the control algorithm to be fully automated, but the delivered therapy may utilize user input in other embodiments. The high-pressure catheter connector and the controller connector facilitate the liquid connection when both are securely locked together. Once securely locked together, the treatment motor traverses the cryogenic delivery channels within the probe's predefined configuration. The exhaust valve controls are fluidly coupled to the catheter and control the gas outlet from the probe and into the exhausting chamber. The controller can be configured to automatically control the outlet of gas from the probe through the exhaust valve before, during, and after the cryogenic fluid is flowing through the probe. Once the cryogen fluid has converted into a gas within the probe, it is conveyed back into the controller through the shaft inner space (FIG. 11 and FIG. 12). It travels through the handle connector, exhaust valve, and into the exhaust chamber. Any remaining cryogen left within the cartridge after completing the therapy may be vented from the cartridge. The cartridge's venting conveys the cryogen fluid directly into the exhaust chamber. Once the cryogen has been contained within the exhaust chamber, it can be directed from the controller in several different ways. The exhaust from the system can be configured for direct or recovery of the gas.

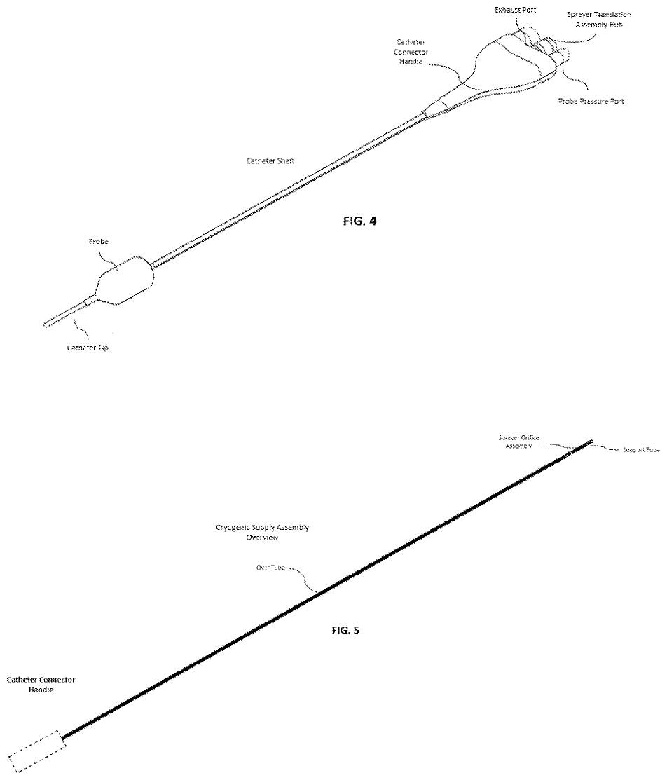

Referring to FIG. 4, is an embodiment of a perspective view of the catheter constructed according to the present invention's teachings and has a distal end and a proximal end. The proximal end of the catheter carries a catheter connector handle, through which the catheter is securely received into the controller.

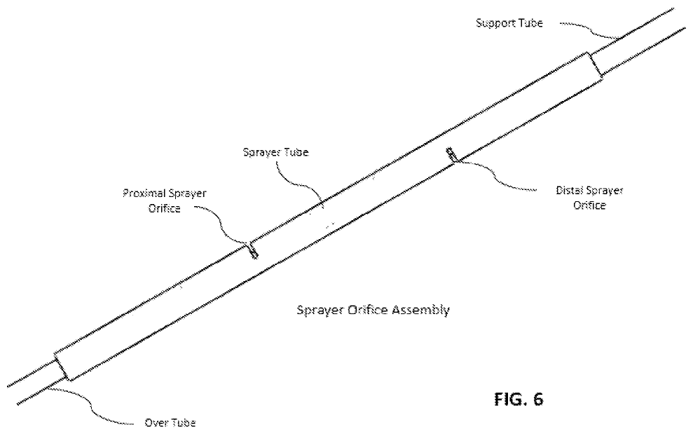

Referring to FIG. 5 and FIG. 6 is an embodiment of a perspective view of the cryogenic supply assembly inside the probe where the probe is omitted for clarity. The cryogenic supply assembly components deliver the cryogenic fluid from the catheter connector handle to the probe. The cryogenic supply assembly consists of the cryogenic supply line, over tube, and sprayer orifice assembly. The cryogenic fluid flows to the probe through the cryogenic supply line located within the cryogenic supply assembly. When the catheter connector handle is installed within the controller, the cryogenic supply line is fluidly coupled to the controller through the high-pressure port. It provides the conduit for the cryogenic fluid to flow to the probe.

When the cryogenic fluid flows to the sprayer orifice assembly, as shown in FIG. 22A, FIG. 22B, FIG. 23A, and FIG. 23B, within the probe, the cryogenic fluid must exit the cryogenic supply line and enter the sprayer orifice assembly precisely within one of the seal chambers containing a sprayer orifice. A hole or series of holes through a single or double wall of the cryogenic supply line provides a pathway for the cryogenic fluid to travel through and into the sprayer orifice assembly. The hole must be equal to or greater than the 20) volume of the diameter of the cryogenic supply line. The cryogenic supply line must tolerate the pressures and withstand the chemical properties associated with cryogenic fluids. The cryogenic supply line can be from materials such as metals, alloys, thermoplastic, or thermoset plastic that include but are not limited to stainless steel, aluminum, nitinol, Pebax, PET, PEEK, nylon, and Polyimide.

Referring to FIG. 7A, an embodiment of the cryogenic supply line, can be manufactured from a solid tube.

Referring to FIG. 7B, an embodiment of the cryogenic supply line can be manufactured from Polyimide to include a braiding, and/or axial reinforcement wires that run through the length of the tubing. The construction of a reinforced tube consists of a substrate layer, braid and/or axial reinforcement layer, and the exterior layer. Additionally, different coatings can be applied to the exterior and/or interior of the tube that can provide lubricity to reduce surface friction and/or to improve chemical and/or thermal bonding of other components to the tubing. The braiding and axial reinforcement can be round wires and/or flat ribbon in various diameters and/or sizes. Primary advantages of braiding in the polyimide cryogenic supply line include increased column strength, torque transmission, and structural support: to ease bending stress and reduce kinking while making aggressive, high angle turns. The braiding density is determined by measuring the distance between each time the wires and/or ribbon cross. The braiding per inch cross (PIC) count of at least 20 to 150 is found to be an acceptable range. To provide variable flexibility to the cryogenic supply line, the per inch cross (PIC) count can be altered and varied within a specific length of the cryogenic supply line. A lower per inch cross (PIC) count increases longitudinal stiffness, while a higher per inch cross (PIC) count tends to improve flexibility. This can create a tube with varying degrees of flexibility and stiffness along its length.

Referring to FIG. 8, an embodiment of the braided cryogenic supply line with a stiffer section near the catheter connector handle while providing a more flexible section near the catheter tip. The axial reinforcement may be woven within the braid or run separately, providing axial reinforcement, and preventing elongation of the native polyimide tubing. Be woven within the braid or run separately, providing axial reinforcement, and preventing the native polyimide tubing elongation. An unconstrained polyimide tube when cryogenic fluid is released within will elongate over time.

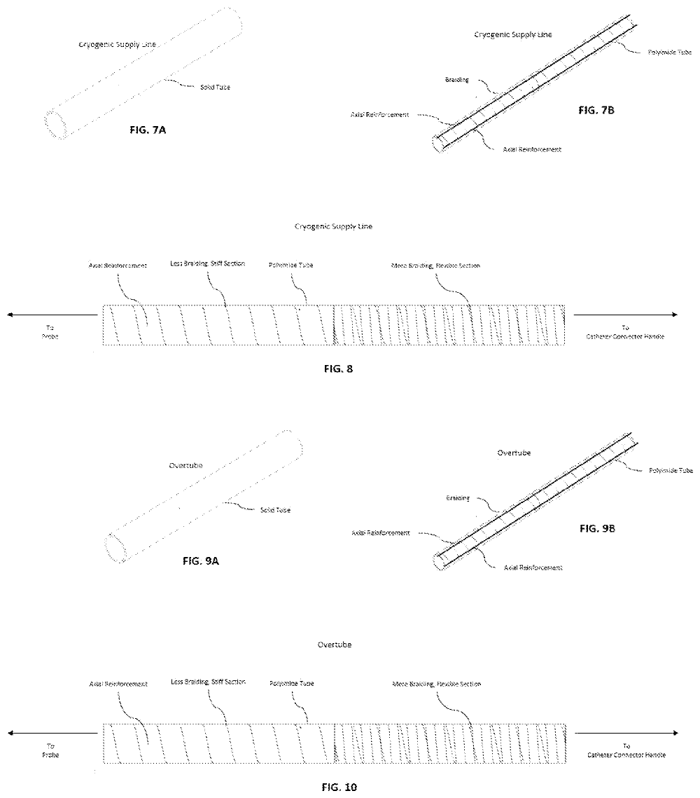

Referring to FIG. 9A and FIG. 9B, the over tube provides axial support for the sprayer orifice assembly when the cryogenic supply line moves within. The over tube is secured within the sprayer selector portion of the catheter connector handle and runs through the catheter connector handle and into the catheter shaft, and then into the probe. The over tube must provide linear support for the sprayer orifice assembly to reduce and/or prevent compressive and elongation forces during use. The over tube can be from materials such as metals, alloys, thermoplastic, and/or thermoset plastic that include but are not limited to Stainless steel, Aluminum, Nitinol, Pebax. PET. PEEK, Nylon, and Polyimide.

Referring to FIG. 9A, an embodiment of the over tube, can be manufactured from one and/or several different materials creating a solid tube.

Referring to FIG. 9B, an embodiment of the over tube, can be manufactured from Polyimide to include a braiding and/or axial reinforcement that runs through the tubing length. The construction of a reinforced over tube consists of a substrate layer, braid and/or axial reinforcement layer, and the exterior layer. Additionally different coatings may be applied to the exterior and/or interior of the tube that can provide lubricity to reduce surface friction or thermoplastic materials to improve chemical and/or thermal bonding of other components to the over tube. The braiding and/or axial reinforcement can be round wires and/or flat ribbon in various diameters and/or sizes. Primary advantages of braiding in the polyimide cryogenic supply line include increased column strength, torque transmission, and structural support; to ease bending stress and reduce kinking while making aggressive, high angle turns. The braiding density is determined by measuring the distance between each time the wires and/or ribbon cross. The braiding per inch cross (PIC) count of at least 20 to 150 is found to be an acceptable range. To provide variable flexibility to the cryogenic supply line, the per inch cross (PIC) count can be altered and varied within a specific length of the over tube. A lower per inch cross (PIC) count increases longitudinal stiffness, while a higher per inch cross (PIC) count tends to improve flexibility. This can create a tube with varying degrees of flexibility and stiffness along its length.

Referring to FIG. 10, an embodiment of the braided over tube with a stiffer section near the catheter connector handle while providing a more flexible section near the probe. The axial reinforcement may be woven within the braid or run separate, providing axial reinforcement, and preventing the native polyimide tubing elongation.

Referring to FIG. 11, an embodiment of a cross-sectional view of the catheter shaft. This configuration is a tri-lumen extrusion tube providing individual conduits that are incorporated into the catheter shaft wall that can be used to monitor the probe pressure and remove gas when the cryogenic fluid undergoes a phase change from liquid to gas and accommodates the cryogenic supply assembly. Due to tortuous anatomy and the precise positioning of the sprayer orifice assembly, the cryogenic supply assembly must be constrained to prevent excessive lateral movement. The cryogenic supply assembly lumen is used to house and guide the cryogenic supply assembly from the catheter connector handle to the probe. The catheter shaft can be selected from a lubricous substance that is flexible and tough. The gas from the probe passes through the catheter shaft's largest inner space, the exhaust lumen. The probe pressure can be incorporated into the lumen located in the catheter shaft wall or ran as a separate tube from the controller to within proximity of the probe. The probe pressure lumen is used to obtain a consistent pressure measurement of the probe.

Referring to FIG. 12, an embodiment of a cross-sectional view of the catheter shaft. This configuration is a tri-lumen extrusion tube providing individual conduits that are incorporated into the catheter shaft wall that can be used to monitor the probe pressure and remove gas when the cryogenic fluid undergoes a phase change from liquid to gas and accommodates the cryogenic supply assembly. Due to tortuous anatomy and the precise positioning of the sprayer orifice assembly, the cryogenic supply assembly must be constrained to prevent excessive lateral movement. The support member elevates the cryogenic supply assembly lumen to keep the cryogenic supply assembly centered within the catheter shaft. The cryogenic supply assembly lumen is used to house and guide the cryogenic supply assembly from the catheter connector handle to the probe. The catheter shaft can be selected from a lubricous substance that is both flexible and tough. The gas from the probe passes through the catheter shaft's largest inner space, the exhaust lumen. The probe pressure can be incorporated into the lumen located in the catheter shaft wall or run as a separate tube from the controller to within proximity of the probe. The probe pressure lumen is used to obtain a consistent pressure measurement of the probe.

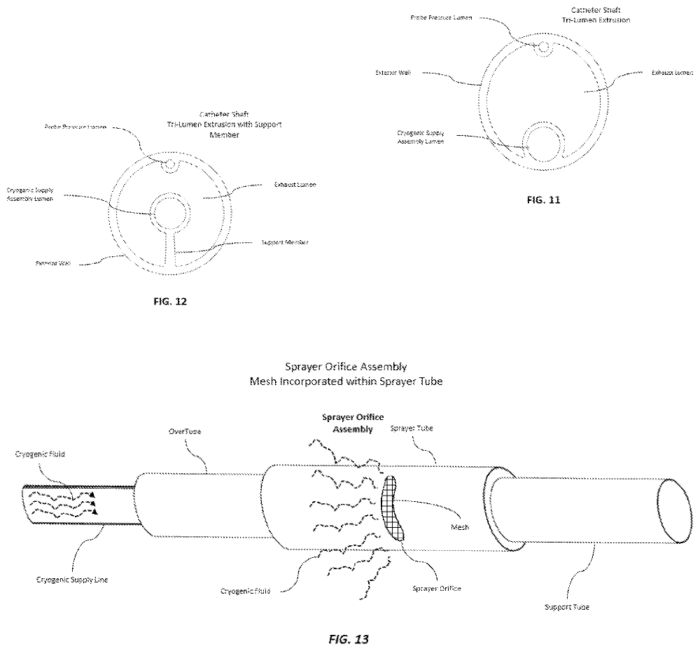

Referring to FIG. 13, FIG. 15, and FIG. 16, several embodiments of the cryogenic supply assembly, includes the sprayer orifice assembly that provides a means for directing and evenly distributing the cryogenic fluid across the sprayer orifice before coming in contact with the active area of the probe. The active area is defined as the area of the probe in intimate contact with the tissue. During use, the sprayer orifice assembly must provide structural support for the sprayer orifices, seal chambers, and cryogenic supply line.

The sprayer tube can be made from materials such as metals, alloys, or thermoset plastic that include but are not limited to, Stainless steel, Aluminum, Nitinol. and Polyimide. Additional materials that can withstand both the bending stresses associated within the anatomy as well as the chemical and physical properties of cryogenic fluids. The sprayer orifice design and material must prevent compressive, and elongation forces exerted on the sprayer orifice from changing its shape and/or profile during use. The material must accommodate various shapes of holes that create a pattern to form the sprayer orifice for the cryogenic fluid to pass through.

Referring to FIG. 13, an embodiment of the sprayer orifice assembly that provides a means for directing and evenly distributing the cryogenic fluid across the sprayer orifice before coming in contact with the active area of the probe. The material as shown for this sprayer orifice assembly is Polyimide, although other materials could be used to provide a similar result.

This material can be manufactured to include a braiding, and/or axial reinforcement that runs through the length of the tubing. The construction of a reinforced tube consists of a substrate layer, braid and/or axial reinforcement layer and the exterior layer. The substrate and exterior layers are polyimide encase the braiding within. Additionally, different coatings may be applied to the tube's exterior and/or interior that can provide lubricity to reduce surface friction and/or thermoplastic materials to improve chemical and/or thermal bonding of other components to the tubing. The braiding and axial reinforcement can be round wires and/or flat ribbon in various diameters and/or sizes.

Primary advantages of using braiding in the sprayer tube include increased column strength, torque transmission, and structural support: to ease bending stress and reduce kinking while making aggressive, high angle turns. The braiding density is determined by measuring the distance between each time the wires cross. The braiding per inch cross (PIC) count of at least 100 to 300 is found to be an acceptable range. A lower per inch cross (PIC) count tends to increase longitudinal stiffness, while a higher per inch cross (PIC) count tends to improve flexibility.

The axial reinforcement may be woven within the braid or run separately, providing axial reinforcement, and preventing the native polyimide tubing elongation. The sprayer orifice is defined by various shapes of hole(s) that create a pattern that forms the sprayer orifice for the cryogenic fluid to pass through, including but are not limited to circle(s), oval(s), triangle(s), square(s), rectangle(s), diamond(s), hexagon(s), and octagon(s) or some combination of shapes creating a unique shape. The removal of Polyimide from around the braiding makes both the sprayer orifice for the cryogenic fluid to pass through and the structural support for the sprayer orifices.

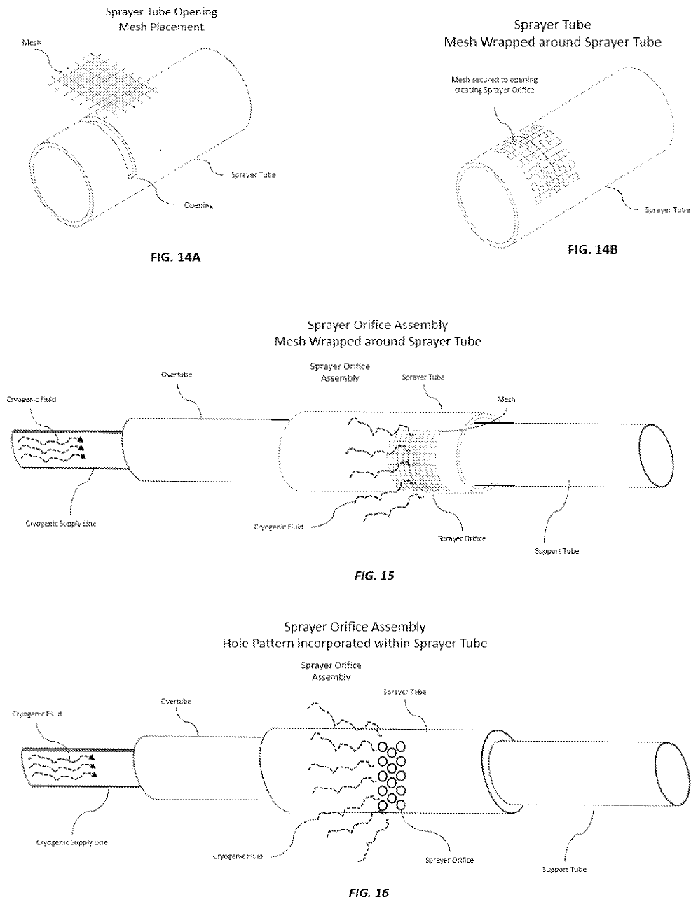

Referring to FIG. 14A, an embodiment of the sprayer tube that utilizes a rigid tube with a sprayer opening that incorporates a mesh wrapped and secured as shown in FIG. 14B, securing it around the sprayer orifice. The sprayer tube can be made from several different materials such as, Stainless Steels, Aluminum, and Nitinol. The mesh can be made from several different materials such as, Stainless Steels, Plain and/or coated Steels, Aluminum. Plastics. A variety of coatings can be applied to the mesh such as, PTFE, Poly, Powder, Galvanize. The mesh can be either welded, woven, or sintered. The specifications used to define the mesh is mesh size, wire diameter, opening area. The mesh size is how many wires per inch are within the mesh. The diameter of the wire defines the size of each individual wire used that make up the mesh. While the open area defines the size of the open area between each wire strand. A mesh size of at least 100 to 400 is found to be an acceptable range. The mesh must be flexible enough to wrap around the sprayer tube while providing structural support for the sprayer orifice.

Referring to FIG. 15, an embodiment of mesh over the sprayer orifice assembly creates the geometry required for even distribution of the cryogenic fluid across the sprayer orifice. The physical properties of the mesh and the way its secured around the sprayer tube creates the support for the sprayer orifice during use. The mesh can be secured to the sprayer tube using adhesive, welding, slightly larger diameter tube cut to form a window and/or slot opening around the sprayer orifice and/or a combination of these techniques to secure the mesh to the sprayer tube to create the necessary geometry required to evenly distribute the cryogenic fluid across the sprayer orifice surface. The sprayer tube opening can be defined by various shapes, a hole and/or holes that create an opening that create a pattern that form the sprayer orifice for the cryogenic fluid to pass through that include but are not limited to, circle(s), oval(s), triangle(s), square(s), rectangle(s), diamond(s), hexagon(s), and octagon(s) or some combination of shapes creating a unique shape.

Referring to FIG. 16 is an embodiment of the sprayer tube utilizing a rigid tube where a series of holes are cut into the tube, forming a pattern that creates the sprayer orifice. This sprayer tube can be made from several different materials such as Stainless steel, Aluminum, and Nitinol. While nitinol is shown other materials for the sprayer tube can be used that exhibit similar properties. These materials must accommodate cutting a series of holes through the sprayer tube creating a passageway for the cryogenic fluid to flow between the interior and exterior of the sprayer tube.

Referring to FIG. 17, an embodiment of the sprayer orifice that is defined by various shapes of hole(s) that create a pattern that form the sprayer orifice for the cryogenic fluid to pass through that include but are not limited to, circle(s), oval(s), triangle(s), square(s), rectangle(s), diamond(s), hexagon(s), and octagon(s) or some combination of shapes creating a unique shape. Each sprayer orifice must be distributed along the axis of the sprayer orifice assembly and contain its own sealing chamber. The cut hole(s) form a pattern that creates both the sprayer orifice for the cryogenic fluid to pass through and the structural support for the sprayer orifice. This technique makes the geometry required to distribute the cryogenic fluid across the sprayer orifice surface evenly.

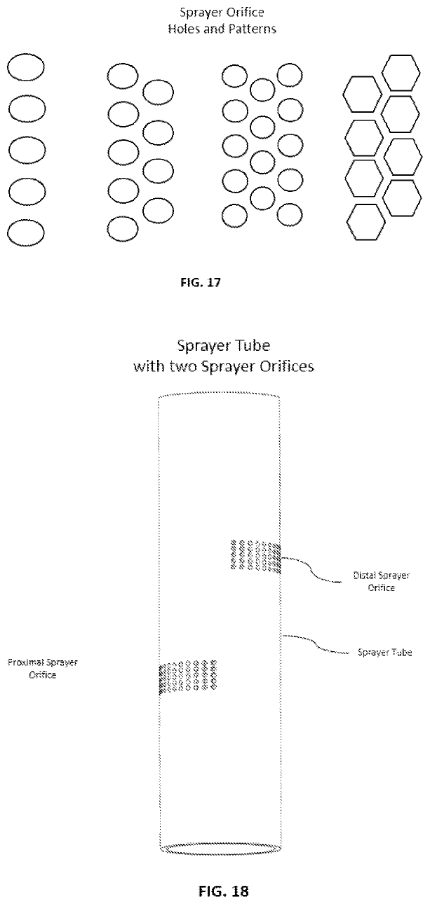

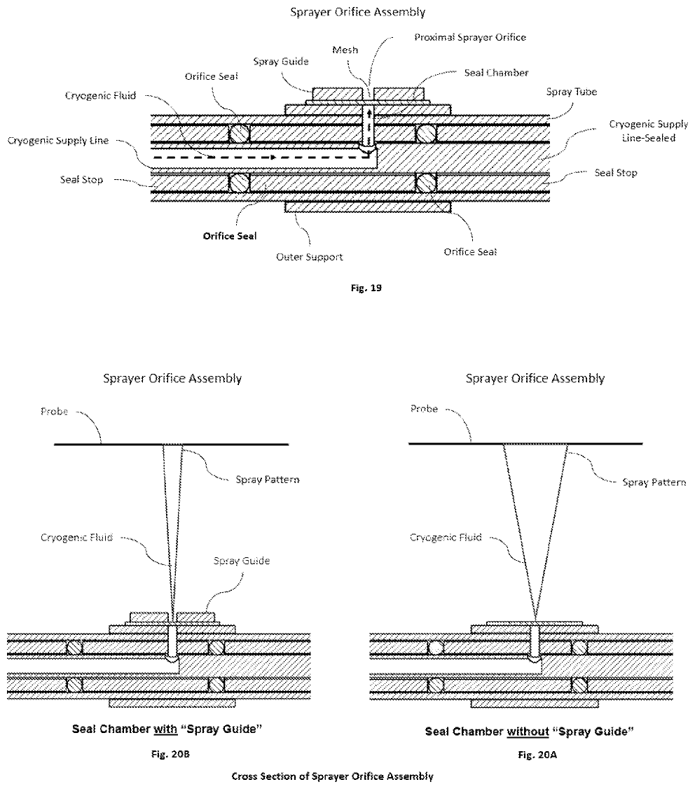

FIG. 18 is an embodiment of the sprayer orifice assembly that contains at least one or a series of sprayer orifices that are distributed around the circumference of the sprayer tube, two are shown. The distribution and the total number of sprayer orifices may have 360 degrees of cryogenic spray coverage and may be designed to achieve a total of 360 degrees of cryogenic spray around the inner surface of the probe or designed for spray overlap or designed for gaps around the circumference of the sprayer tube. The arc length of each sprayer orifice can be designed between 5 and 360 degrees. Although two 180-degree sprayer orifices are shown, at least one to more sprayer orifices is found to be acceptable. Each sprayer orifice may be evenly distributed along the axis of the cryogenic supply assembly and contain its own sealing chamber. The pattern of shapes creating the surface area that makes up each sprayer orifice is designed to evenly distribute cryogenic fluid across the surface of the sprayer orifice. When the fluid is sprayed on the probe's inner surface, a consistent temperature profile is achieved. The maximum temperature variation across the inner probe surface from a single sprayer orifice is within several degrees Celsius.

The sprayer tube is the final channel for the cryogenic fluid before being released into the probe. To evenly distribute the cryogenic fluid across the sprayer orifice a restriction must be created forcing the fluid across the sprayer orifice surface area. The size and shape of the holes that make up the sprayer orifice have a critical role in creating a restriction for the cryogenic fluid.

A single and/or several openings that equal 180 degrees or greater than the diameter of the tube will not create the proper restriction of fluid, and therefore, an inconsistent spray profile is achieved on the inner surface of the probe. Suppose the sprayer orifice is significantly greater in volume with no flow restriction of the cryogenic fluid. In that case, the turbulent cryogenic fluid gets dispersed unevenly across the inner area of the probe.

The even distribution of cryogenic fluid across the sprayer orifice is aided by the creation of a restriction and/or backpressure attributed to the mesh/screen. This restriction evenly distributes the cryogenic fluid across the sprayer tube orifice that contacts the inner surface of the probe.

Referring to FIG. 19 and FIG. 21, a section view of the Sprayer Orifice Assembly, this configuration utilizes an outer support for the sprayer tube, which can be a tube, sleeve, or ribbon. This support tube provides reinforcement for the sprayer tube openings, preventing them from either shrinking or expanding. It can encircle the entire circumference of the sprayer tube, either partially or completely, with a minimum thickness of 0.001 inches. This outer support can be fabricated from a range of materials, including plastics, stainless steel, aluminum, and Nitinol. To affix the tube or sleeve to the sprayer tube, multiple attachment methods are available. Secure attachment can be achieved using adhesive or welding. In the case of a tube, it is possible to select one with a slightly larger diameter and subsequently create an opening that matches the size of the sprayer tube's orifice, either slightly larger or smaller. This effectively forms an enclosure around the sprayer orifice. Moreover, it is possible to combine these attachment techniques as needed to achieve the specific geometry required to provide robust support to the sprayer tube.

Referring to FIG. 19 and FIG. 21 a section view of the Sprayer Orifice Assembly, this configuration utilizes an inner support for the sprayer tube, which can be a tube or sleeve. This support tube minimizes the sealing chamber's internal volume to closely match the CSL tube's cross-sectional area within the sprayer tube. This support tube can be fabricated from a range of materials, including plastics, stainless steel, aluminum, and Nitinol. To affix the tube or sleeve to the sprayer tube or cryogenic supply line, multiple attachment methods are available. Secure attachment can be achieved using adhesive or welding. An opening that matches the size of the cryogenic supply line, either slightly larger or smaller.

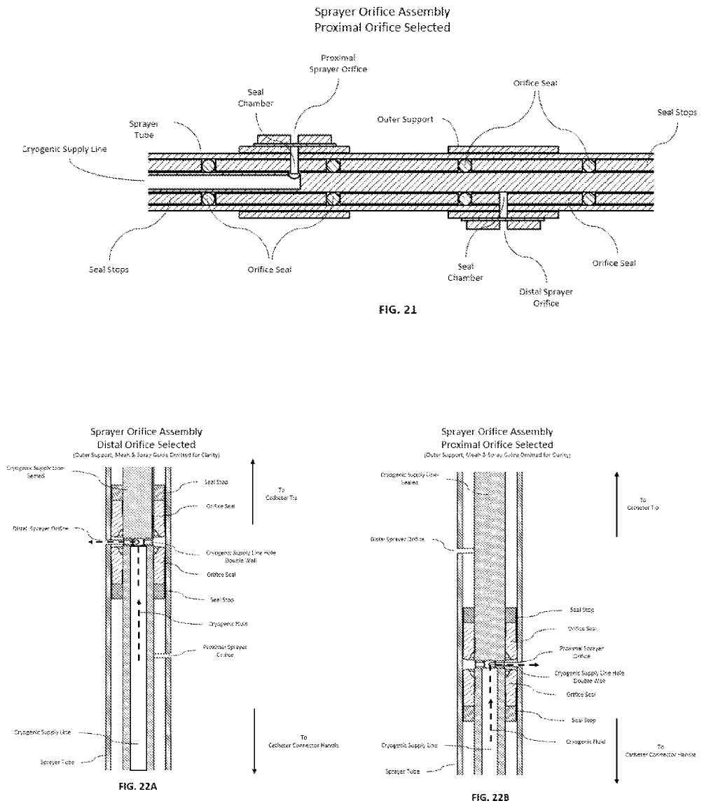

Referring to FIG. 20A and FIG. 20B is a section view of the Sprayer Orifice Assembly this configuration utilizes a spray guide which plays a pivotal role in preventing excessive overspray, thereby ensuring a cleaner spray pattern when the cryogen comes into contact with the inner surface of the probe. Increasing the thickness of the spray guide narrows and sharpens the focus of the spray pattern, allowing for precise adjustment of the cryogen spray pattern. This results in improved spray precision. This adjustment is a key factor in determining the treatment dosage. Moreover, this capacity to modify the spray adds flexibility to the system for fine-tuning the treatment, in conjunction with sharper more focus spray pattern, traversing speed, and flow rate, thus enhancing the precision of the treatment algorithm. To ensure the stability of the spray guide on the sprayer tube, it must be affixed direct on top and in line with the spray tube opening. The spray guide can take the form of a tube, sleeve, or ribbon and is designed to encircle the sprayer opening wrapping the entire circumference of the sprayer tube partially or completely, with a minimum thickness of at least 0.001 inches. This guide can be fabricated from a range of materials, including plastics, stainless steel, aluminum, or Nitinol. Secure attachment can be achieved using adhesive or welding.

Referring to FIG. 19 and FIG. 21, a seal chamber may consist of two or four seal stops, and two or three orifice seals, creating individual seal chambers.

Referring to FIG. 22A and FIG. 22B, an embodiment of the sprayer orifice assembly, consists of the sprayer tube, two orifice seals, and two seal stops. In this configuration, the orifice seals and seal stops are attached to the cryogenic supply line. The cryogenic supply line, orifice seal, and seal stop create an individual seal chamber that can be moved axially within the sprayer tube. The seal chamber can be aligned with the sprayer orifice allowing the cryogenic fluid to flow through an individual sprayer orifice.

Referring to FIGS. 23A and 23B is an embodiment of the sprayer orifice assembly, which consists of three seal stops where two of the stops use the over tube, and the support tube and Six orifice seals create two seal chambers. Two seal stops entrap the orifice seal(s) making a seal chamber. In this configuration, the orifice seals and seal stops are attached to the sprayer tube allowing for independent movement of the cryogenic supply line.

There are two functions the orifice seal must perform when the system is operating. The first is to provide a bubble-tight seal chamber during use when the cryogenic fluid is flowing through the system. The second allows the cryogenic supply line freedom to move axially within the sprayer tube. The volume of the sealing chamber is critical for the correct operation of the sprayer orifice assembly. A sealing chamber that is too small or large will adversely affect the distribution of cryogenic fluid spray onto the inner surface of the probe.

Referring to FIG. 4, an embodiment of the catheter system in the load position where the controller is omitted for clarity. The operational sequence is similar for all configurations, with the expectation of the sprayer selector having one or more positions based on the number of sprayer orifices within the sprayer assembly.

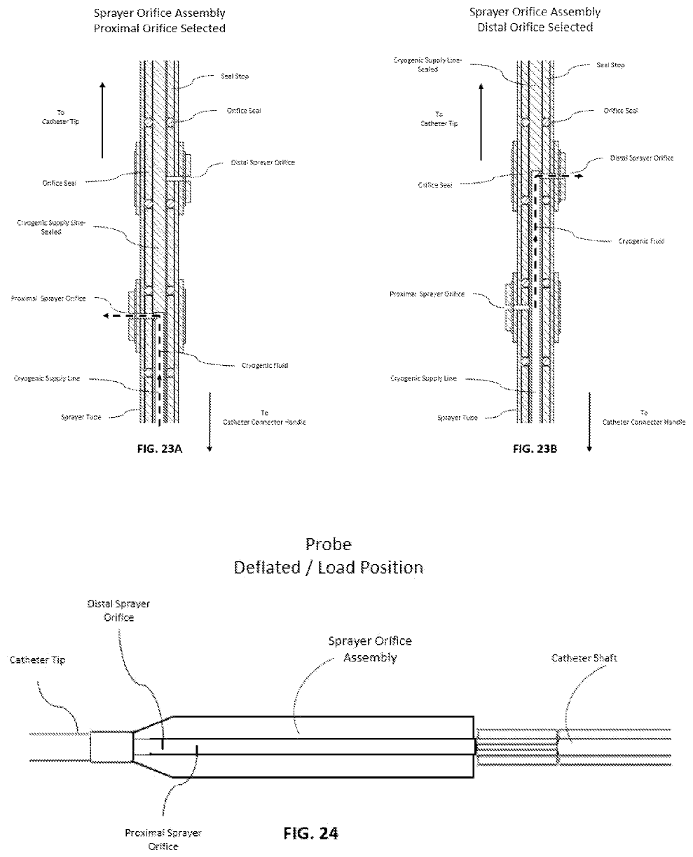

Referring to FIG. 24, the probe is shown deflated. The cryogenic supply assembly is located at the distal-most portion within the probe. In this position, the catheter is inserted into an endoscope, an accessory channel, or inside a delivery sheath for use. After insertion into and through an endoscope or after the endoscope is in place, the user can confirm the placement and location of the probe using the endoscope's visualization capabilities.

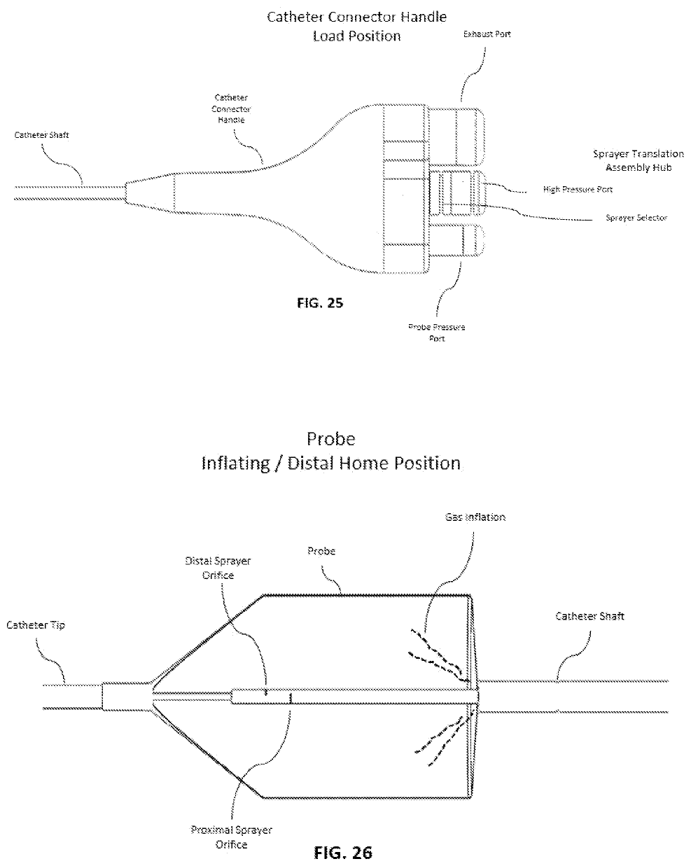

Referring to FIG. 25, a top view of the catheter connector handle in the load position where the catheter connector handle may be inserted into the controller connector, as 30 shown in FIG. 4. The catheter is inserted using the catheter connector handle with the three ports consisting of the probe pressure port, exhaust port, and sprayer translation assembly hub end first into the controller connector until the catheter connector handle stops. The controller automatically detects the catheter's presence and indicates that the catheter is secured and fluidly coupled to the controller via the touch display.

Referring to FIG. 26, the probe is shown inflated, and the sprayer assembly is located at the distal home position within the probe. After the catheter is connected to the controller the user selects to inflate the probe via the touch display. Once the user makes the selection to inflate the catheter probe the controller starts to pressurize the probe utilizing the inflation system while moving the sprayer translation assembly hub to the default start position aligning the distal sprayer orifice with the most distal section of the active area of the probe known as the distal home position. The active area is defined as the area of the probe in intimate contact with the tissue. When in connection with the controller, the cryogenic fluid is being delivered through the sprayer translation assembly hub via the high-pressure port through the sprayer orifice onto the active area of the probe's inner surface.

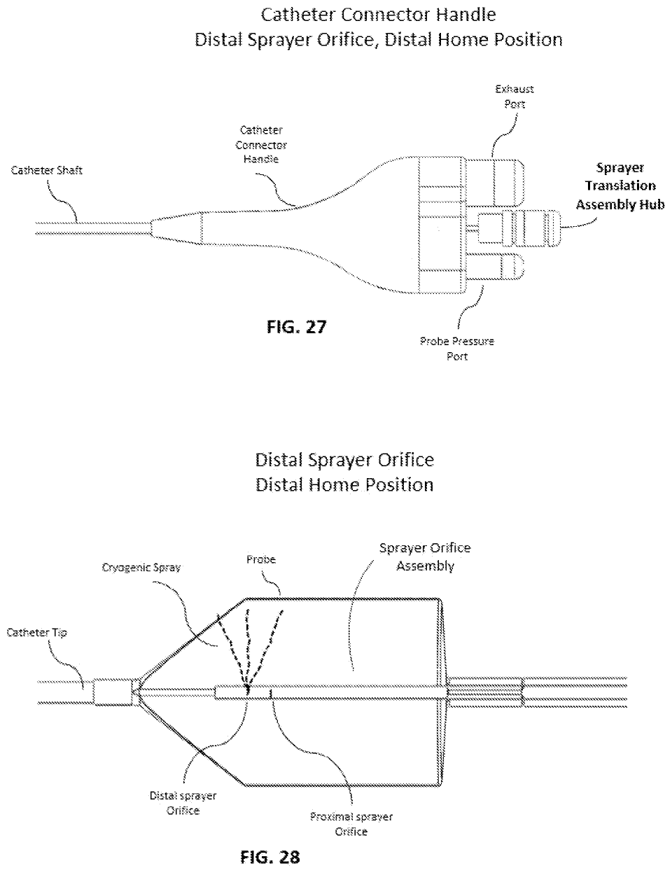

Referring to FIG. 27, an embodiment of the catheter connector handle, is shown where the distal sprayer orifice is selected to deliver cryogenic fluid to the inside of the probe's most distal section of the active area. The controller is omitted for clarity. In this position, the catheter can deliver cryogenic fluid into the probe through the sprayer translation assembly hub.

Referring to FIG. 28, the embodiment of the probe with the distal sprayer orifice selected in the distal home position corresponds to the position of the catheter connector handle shown in FIG. 27. The distal sprayer orifice is positioned in the most distal portion of the active area inside of the probe. When connected with the controller, the cryogenic fluid is being delivered through the sprayer translation assembly hub via the high-pressure port through the sprayer orifice onto the active area of the probe. To increase the ablation area, the sprayer translation assembly hub, in connection with the controller, can move axially, translating the sprayer orifice assembly within the probe. The sprayer orifice assembly consists of the sprayer tube, orifice seals, and seal stops.

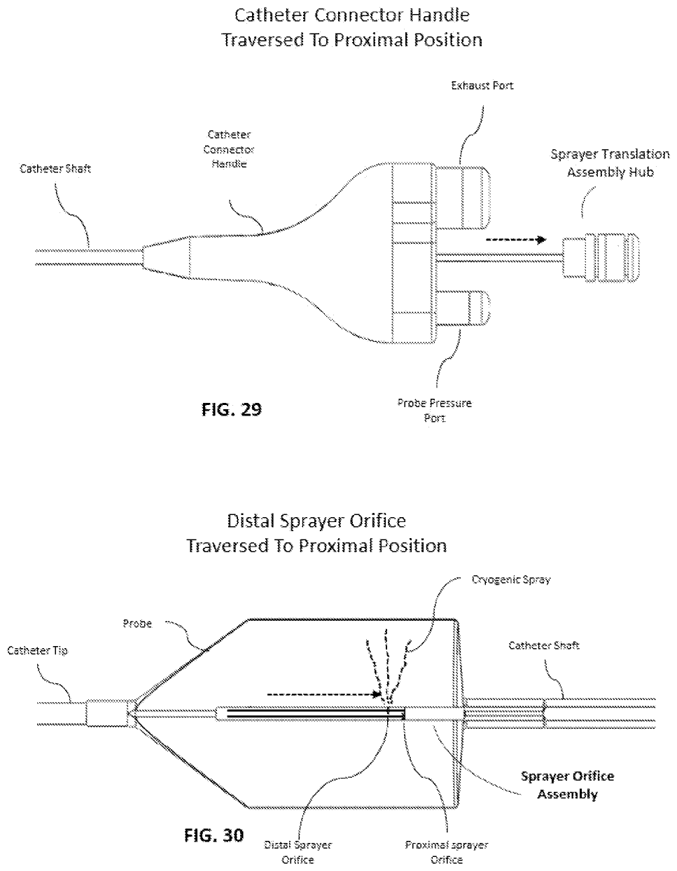

Referring to FIG. 29, the embodiment of the catheter connector handle, shows where the distal sprayer orifice is selected to deliver cryogenic fluid to the active area of the probe, and the controller is omitted for clarity. In this position, the controller can provide cryogenic fluid into the probe through the sprayer translation assembly hub. Axially, the sprayer translation assembly hub is pulled away from the catheter connector handle.

Referring to FIG. 30, the embodiment of the probe with the distal sprayer orifice selected in the proximal position corresponds to the catheter connector handle position shown in FIG. 29. The sprayer translation assembly hub is positioned in the most proximal portion of the active area of the probe. When in connection with the controller, cryogenic fluid is being delivered through the sprayer translation assembly hub via the high-pressure port through the distal sprayer orifice onto the active area of the probe. With the axial translation of the sprayer translation assembly hub, the distal sprayer orifice is now positioned to the most proximal portion of the inside of the probe. The sprayer translation assembly hub may be positioned between the most distal and most proximal portion of the probe delivering cryogenic fluid to any portion of the active area of the probe.

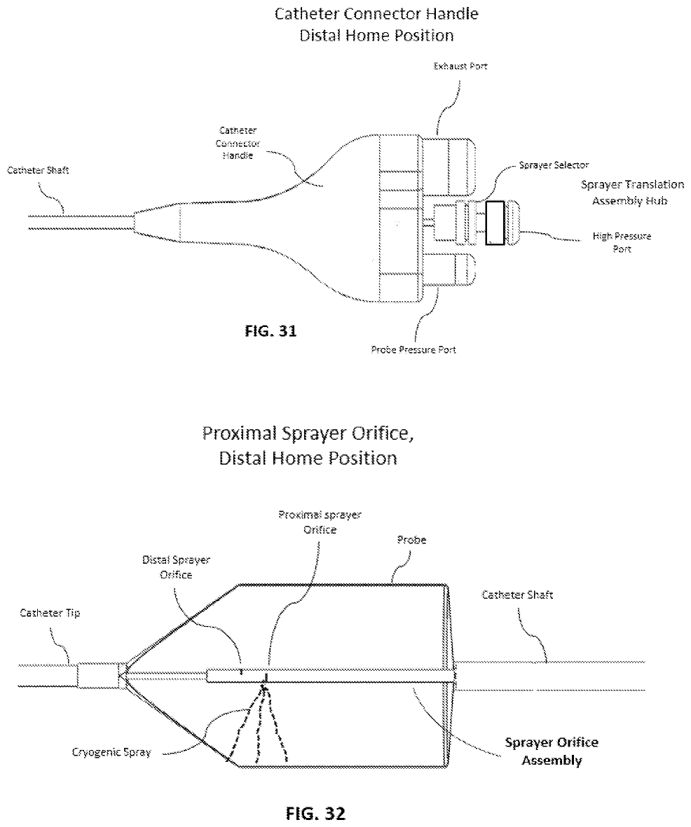

The high-pressure port and the sprayer selector together form the sprayer translation assembly hub. The sprayer translation assembly hub is used for selection of one or more sprayer orifices based on catheter configuration used with the controller. Referring to FIG. 31 is an embodiment of the catheter connector handle, is shown to select the proximal sprayer orifice. The sprayer selector is pulled away, separating it from the high-pressure port when connected with the controller. Referring to FIG. 32, an embodiment of the probe with the proximal sprayer orifice selected in the distal home position corresponds to the catheter connector handle position shown in FIG. 31.

Referring to FIG. 31, an embodiment of the catheter connector handle, is shown where the proximal sprayer orifice is selected to deliver cryogenic fluid to the inside of the probe's most distal section of the active area, and the controller is omitted for clarity. In this position, the controller can deliver cryogenic fluid into the probe through the sprayer translation assembly hub.

Referring to FIG. 32, the embodiment of the probe with the proximal sprayer orifice selected in the distal home position corresponds to the position of the catheter connector handle shown in FIG. 31. The proximal sprayer orifice is positioned in the most distal portion of the active area inside of the probe. When in connection with the controller, the cryogenic fluid is being delivered through the sprayer translation assembly hub via the high-pressure port through the sprayer orifice onto the active area of the probe. To increase the ablation area, the sprayer translation assembly hub, in connection with the controller, can move axially, translating the sprayer orifice assembly within the probe.

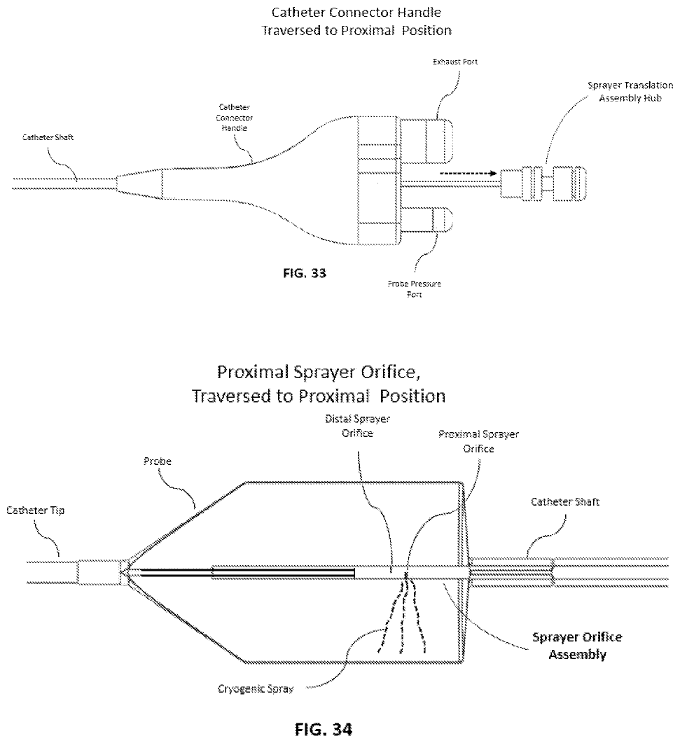

Referring to FIG. 33, the embodiment of the catheter connector handle, shows where the proximal sprayer orifice is selected to deliver cryogenic fluid to the active area of the probe, and the controller is omitted for clarity. In this position, the controller can deliver cryogenic fluid into the probe through the sprayer translation assembly hub. The sprayer translation assembly hub is pulled away from the catheter connector handle axially.

Referring to FIG. 34, the embodiment of the probe with the proximal orifice selected in the proximal position corresponds to the position of the catheter connector handle shown in FIG. 34 The sprayer translation assembly hub is positioned in the most proximal portion of the active area of the probe. When in connection with the controller cryogenic fluid is being delivered through the sprayer translation assembly hub via the high-pressure port through the proximal sprayer orifice onto the active area of the probe. With the axial translation of the sprayer translation assembly hub, the proximal sprayer is now positioned to the most proximal portion of the active area of the probe. The sprayer translation assembly hub may be positioned between the most distal and most proximal portion of the probe delivering cryogenic fluid to any portion of the active area of the probe.

Table B is a listing of certain of the various elements or items shown in the as-filed Figures, along with certain embodiments of the elements or items suitable for use with the catheters, systems, and methods of the present disclosure. The embodiments described in Table B are not meant to limit the scope of the catheters, systems, and methods of the present disclosure.

TABLE B

| Ref. | | | |
|---|---|---|---|
| No. | Element/Item | FIGS. | Embodiments |
| 1 | Agil's Cryoablation | 2, 7B, 8, 9B, 10 | |
| 2 | Axial Reinforcement | 7B, 8, 9B, 10 | Ø 0.001″ to 0.005″ × 0.060″ to |
| | | | 120.0″ Length |
| 3 | Braiding | 7B, 9B | See Overtube and Cryogenic |
| | | | Supply Line |
| 4 | Cartridge Container | 3 | Custom Machined or Molded |
| 5 | Catheter | 2 | |
| 6 | Catheter Connector | 4, 5, 25, 27, 29, | Custom Machined or Molded |
| | Handle | 31, 33 | |
| 7 | Catheter Locking Motor | 3 | Off the Shelf Motor |
| 8 | Catheter Shaft | 4, 24, 25, 26, 27, | Custom Extrusion - 60.0″ to |
| | | 28, 29, 30, 31, 32, | 120.0″ length × 0.030″ to 0.080″ |
| | | 33, 34 | Main ID, 0.010″ × 0.060″ Next |
| | | | Largest ID × 0.002″ to 0.020″ |
| | | | Smallest ID, 0.002″ 0.030″ walls |
| 9 | Catheter Shaft Tri- | 11 | Custom Extrusion - 60.0″ to |
| | Lumen Extrusion | | 120.0″ length × 0.030″ to 0.080″ |
| | | | Main ID, 0.010″ × 0.060″ Next |
| | | | Largest ID × 0.002″ to 0.020″ |
| | | | Smallest ID, 0.002″ to 0.030″ |
| | | | Walls |
| 10 | Catheter Shaft Tri- | 12 | Custom Extrusion - 60.0″ to |
| | Lumen Extrusion with | | 120.0″ length × 0.030″ to 0.080″ |
| | Support Member | | Main ID, 0.010″ × 0.060″ Next |
| | | | Largest ID × 0.002″ to 0.020″ |
| | | | Smallest ID × 0.002″ × 0.015″ |
| | | | Web |
| 11 | Catheter Tip | 4, 24, 30, 32, 34, | Custom Extrusion - 0.005″ to |
| | | 26, 28 | 0.030″ × 1.0″ to 4.0″ Length |
| 12 | Controller | 1 | |
| 13 | Controller Connector | 1, 3 | Custom Machined or Molded |
| | | | Component |
| 14 | Cryogenic Fluid | 13, 15, 16, 20A, | Off the Shelf Component |
| | | 20B, 22A, 22B, | |
| | | 23A, 23B | |
| 15 | Cryogenic Fluid Deliver | 3 | |
| | System | | |
| 16 | Cryogenic Spray | 28 | |
| 17 | Cryogenic Supply | 11, 12 | See Catheter Shaft, Catheter |
| | Assembly Lumen | | Shaft Tri-Lumen Extrusion or |
| | | | Catheter Shaft Tri-Lumen |
| | | | Extrusion with Support Member |
| 18 | Cryogenic Supply | 5 | |
| | Assembly Overview | | |
| 19 | Cryogenic Supply Block | 3 | Custom Machined or Molded |
| | | | Component |
| 20 | Cryogenic Supply Line | 7A, 7B, 8, 13, 15, | Custom Tube - 0.002″ to 0.020″ |
| | | 16, 19, 21, 22A, | ID × 0.001″ 0.020″ Wall × 70.0″ |
| | | 22B, 23A, 23B | to 120.0″ Length |
| 21 | Cryogenic Supply Line | 22A, 22B | See Cryogenic Supply Line |
| | Hole Double Wall | | |
| 22 | Cryogenic Supply Line | 22B | See Cryogenic Supply Line |
| | Hole Double Wall | | |
| 23 | Cryogenic Supply Line | 22A, 22B | |
| | Sealed | | |
| 24 | Cryogenic Supply | 3 | |
| | System | | |
| 25 | Cryogenic Supply | 16 | |
| | Assembly Hole Pattern | | |
| | Incorporated within | | |
| | Sprayer Tube | | |
| 26 | Cryogenic Supply | 13 | |
| | Assembly Mesh | | |
| | Incorporated within | | |
| | Sprayer Tube | | |
| 27 | Cryogenic Supply | 15 | |
| | Assembly Mesh | | |
| | Wrapped around Sprayer | | |
| | Tube | | |
| 28 | Delivery Channel Flow | 3 | Off the shelf Valve |
| | Valve | | |
| 29 | Distal Home Position | 27, 28, 31, 32 | |
| 30 | Distal Orifice Selected | 22A | See Opening |
| 31 | Distal Sprayer Orifice | 6, 18, 21, 22A, | See Opening |
| | | 2B, 23A, 23B, 24, | |
| | | 26, 28, 30, 32, 34 | |
| 32 | Duodenum | 2 | |
| 33 | Exhaust Port | 4, 25, 27, 29, 31, | Custom Machined or Molded |
| | | 33 | Component |
| 34 | Exhaust Chamber | 3 | |
| 35 | Exhaust Lumen | 11, 12 | See Catheter Shaft, Catheter |
| | | | Shaft Tri-Lumen Extrusion or |
| | | | Catheter Shaft Tri-Lumen |
| | | | Extrusion with Support Member |
| 36 | Exhaust Valve | 3 | Off the shelf Valve |
| 37 | Exterior Wall | 11, 12 | See Catheter Shaft, Catheter |
| | | | Shaft Tri-Lumen Extrusion or |
| | | | Catheter Shaft Tri-Lumen |
| | | | Extrusion with Support Member |
| 38 | Flow/Pressure PCB | 3 | Off the Shelf Transducer |
| 39 | Gas Inflation | 26 | |
| 40 | Handle | 1 | Custom Machined or Molded |
| | | | Component |
| 41 | High Pressure Connector | 3 | Custom Machined or Molded |
| | | | Component |
| 42 | High Pressure Port | 25, 27, 29, 31 | Custom Machined or Molded |
| | | | Component |
| 43 | Inflating/Distal Home | 26 | |
| | Position | | |
| 44 | Less Braiding, Stiff | 8, 10 | |
| | Section | | |
| 45 | Load Position | 25 | |
| 46 | Main Flow Valve | 3 | Off the Shelf Valve |
| 47 | Mesh | 13, 14A, 15, 19 | Braided Mesh PIC of 20 to 150, |
| | | | 0.010 to 0.050″ W × 0.010″ × |
| | | | 0.2″ L |
| 48 | Mesh secured to opening | 14B | |
| | creating Sprayer Orifice | | |
| 49 | More Braiding, Flexible | 8, 10 | |
| | Section | | |
| 50 | Opening | 14A | Opening - 0.003″ to 0.012″ W × |
| | | | 1° to 360° |
| 51 | Orifice Seal | 19, 21, 22A, 22B, | MFG - O-ring Supplier - Off the |
| | | 23A, 23B | Shelf O-rings |
| 52 | Outer Support | 19, 21 | Custom Extrusion - 0.010″ to |
| | | | 0.0750″ ID × 0.020″ 0.085″ |
| | | | OD × .010″-.125″ Length |
| 53 | Overtube | 5, 6, 9A, 9B, 10, | Custom Tube - 70.0″ to 120.0″ |
| | | 13, 15, 16 | Length × 0.015″ to 0.050″ ID × |
| | | | 0.030″ × 0.065″ OD |
| 54 | Polyimide Tube | 7B, 8, 9B, 10 | See Overtube or Cryogenic |
| | | | Supply Line |
| 55 | Probe | 4, 20A, 20B, 26, | Custom Extrusion, Blown to a |
| | | 28, 30, 32, 34 | diameter of 18 mm to 30 mm × |
| | | | 0.001″ to 0.020″ double wall |
| 56 | Probe Pressure Lumen | 11, 12 | See Catheter Shaft, Catheter |
| | | | Shaft Tri-Lumen Extrusion or |
| | | | Catheter Shaft Tri-Lumen |
| | | | Extrusion with Support Member |
| 57 | Probe Pressure Port | 4, 25, 27, 29, 31, | See Catheter Shaft, Catheter |
| | | 33 | Shaft Tri-Lumen Extrusion or |
| | | | Catheter Shaft Tri-Lumen |
| | | | Extrusion with Support Member |
| 58 | Probe Proximal Orifice | 34 | |
| 59 | Proximal Orifice | 22B | |
| | Selected | | |
| 60 | Proximal Sprayer Orifice | 6, 18, 19, 21, | |
| | | 22A, 22B, 23A, | |
| | | 23B, 24, 26, 28, | |
| | | 30, 32, 34 | |
| 61 | Seal Chamber | 19, 21 | |
| 62 | Seal Stop | 19, 21, 22A, 22B, | Custom Extrusion - 0.005″ to |
| | | 23A, 23B | 0.050″ ID × 0.015″ to 0.060″ |
| | | | OD × 0.005″ to 0.050″ Length |
| 63 | Solid Tube | 7A | See Overtube or Cryogenic |
| | | | Supply Line. |
| 64 | Spray Guide | 19, 20B | Custom Extrusion - 0.030″ to |
| | | | 0.070″ × 0.021″ to 0.086″ ID × |
| | | | 0.001″ to 0.125″ Wall × 0.003″ |
| | | | to 0.1″ Length |
| 65 | Spray Pattern | 20A, 20B | |
| 66 | Sprayer Orifice | 13, 15, 16 | See Opening |
| 67 | Sprayer Orifice | 5, 6, 13, 15, 16, | |
| | Assembly | 19, 20A, 20B, 21, | |
| | | 22A, 22B, 23A, | |
| | | 23B, 24, 28, 30, | |
| | | 32, 34 | |
| 68 | Sprayer Orifice Holes | 17 | Custom Machined or Cut Process |
| | and Patterns | | |
| 69 | Sprayer Selector | 25, 27, 29, 31 | Custom Machined or Molded |
| | | | Component |
| 70 | Sprayer Translation | 4, 25, 27, 29, 31, | See High Pressure Connector or |
| | Assembly Hub | 33 | Sprayer Selector |
| 71 | Sprayer Tube | 6, 13, 14A, 14B, | Custom Tube - 0.009″ to |
| | | 15, 16, 18, 19, 21, | 0.0740″ ID × 0.014″ 0.079″ |
| | | 22A, 22B, 23A, | OD × .010″-120.0″ Length |
| | | 23B | |
| 72 | Sprayer Tube Mesh | 14B | |
| | Wrapped around Sprayer | | |
| | Tube | | |
| 73 | Sprayer Tube Opening | 14A | |
| | Mesh Placement | | |
| 74 | Sprayer Tube with two | 18 | |
| | Sprayer Orifices | | |
| 75 | Stomach | 2 | |
| 76 | Support Member | 12 | See Catheter Shaft |
| 77 | Support Tube | 5, 6 13, 15, 16 | Custom Tube - 0.005″ to 0.050″ |
| | | | ID × 0.015″ to 0.060″ OD × |
| | | | 0.125″ to 6.0″ Length |
| 78 | Traversed to Proxima | 29, 30, 33, 34 | |
| | Position | | |
| 79 | Treatment Motor | 3 | Off the Shelf Motor |

Table C describes certain features and aspects of the cryogenic supply assembly of the present disclosure. The embodiments described in Table C are not meant to limit the scope of the catheters, systems, and methods of the present disclosure.

TABLE C
Cryogenic Supply Assembly
(Example of Certain Embodiments))

| | Features | Description of Embodiments |
|---|---|---|
| Sprayer | Sprayer Name | Sprayer Orifice |
| Portion | # of Sprayers | Multiple |
| | # of openings to achieve | Single, Continuous Opening |
| | spray pattern | |
| | Spray Distribution Across | Liquid (No Phase Change) |
| | Opening | |
| | Mechanism for distribution of | Restricted/Back Pressure/Mesh |
| | cryogen across opening | |
| | Continuous Opening (spray | Yes |
| | Pattern) | |
| | Axial Sprayer Selection | Yes |
| | Activated | Each sprayer is individually on or off based on |
| | | location of CSL, Seal Chamber, or Sprayer Tube. |
| | | Selective ablation is possible (e.g., Activating and |
| | | Deactivating Sprayer Openings). |
| Chamber | Cryogen Chamber Name | Seal Chamber |
| Component | # of Chambers | Single or Multiple |
| | | (Depends on catheter, e.g., 2 at 180°) |
| | Chamber Size | Same Volume as CSL |
| | Communication with | The cryogenic supply line (CSL) is in direct |
| | Delivery Line/CSL | communication with the seal chamber and both |
| | | can be selectively positioned for direct |
| | | communication with a sprayer orifice or the |
| | | cryogenic supply line (CSL) is selectively |
| | | positioned to be direct communication with the |
| | | seal chamber. All can translate as one during |
| | | ablation. |
| Delivery | Cryogen Supply Name | Cryogenic Supply Line |
| Line | # of lines | 1 |
| | Communication | CSL can translate independent of Seal Chamber, |
| | | Sprayer Tube/Sprayer Orifice. CSL can translate |
| | | as a unit with all of above. |
| Treatment | Treatment Targeting | Move CSL and Overtube Axial/Move CSL to |
| | | Select Sprayer |
| | Treat 360° of tissue | Move CSL axially to select different radial |
| | | sprayers |
| | Spray Overlap | Controlled, No rotation |
| | # of sprayers to treat tissue | Single/Multiple |
| | | (Depends on catheter, e.g., 2 at 180°) |

### REFERENCES

Citation of a reference herein shall not be construed as an admission that such reference is prior art to the present invention. All references cited herein are hereby incorporated by reference in their entirety. Below is a listing of various references cited with respect to this example:
1. www.betterhealth.vic.gov.au/health/conditionsandtreatments/diabetes-long-term-effects
2. Liebl A, Mata M, Eschwege E. Evaluation of risk factors for development of complications in Type II diabetes in Europe. Diabetologia 2002;45: S23-S28.
3. Sjöström L, Lindroos A K, Peltonen M, et al. Lifestyle, diabetes, and cardiovascular risk factors 10 years after bariatric surgery. N Engl J Med 2004:351:2683-93.
4. Pories W J, Albrecht R J: Etiology of type 2 diabetes mellitus: role of the foregut. World J Surg 25:527-531, 2001
5. Betzel B, Cooiman M I, Aarts E O, Janssen I M C, Wahab P J, Groenen M J M, Drenth J P H, Berends F J. Clinical follow-up on weight loss, glycemic control, and safety aspects of 24 months of duodenal-jejunal bypass liner implantation. Surg Endosc. 2020 Jan; 34 (1): 209-215.
6. Zervos E E, Agle S C, Warren A J. Lang C G, Fitzgerald T L, Dar M, Rotondo M F, Pories W J. Amelioration of insulin requirement in patients undergoing duodenal bypass for reasons other than obesity implicates foregut factors in the pathophysiology of type II diabetes. J Am Coll Surg. 2010 May: 210 (5): 564-72, 572-4. doi: 10.1016/j.jamcollsurg.2009.12.025. PMID: 20421005.
7 Cherrington A D, Rajagopalan H. Maggs D, Devière J. Hydrothermal Duodenal Mucosal Resurfacing: Role in the Treatment of Metabolic Disease. Gastrointest Endosc Clin N Am. 2017 April: 27 (2): 299-311. doi: 10.1016/j.giec.2016.12.002. PMID: 28292408.
8. van Baar A C G. Holleman F, Crenier L, et al. Endoscopic duodenal mucosal resurfacing for the treatment of type 2 diabetes mellitus: one year results from the first international, open-label, prospective, multicentre study. Gut. 2020: 69 (2): 295-303. doi: 10.1136/gutjnl-2019-318349.
9. ClinicalTrials.gov Identifier—NCT03390322 (clinicaltrials.gov/ct2/show/NCT03390322)
10. Reserved
11. Reserved
12. van Baar A C G, Nieuwdorp M, Holleman F, Soeters M R, Groen A K, Bergman JJGHM. The Duodenum harbors a Broad Untapped Therapeutic Potential. Gastroenterology. 2018 March: 154 (4): 773-777-
13. Dhaliwal_Mo1275 Comparative Assessment of the Structural and Functional Integrity of the Neo-Squamous Epithelium following endoscopic therapy in Barrett's esophagus: A Pilot Study, GIE, POSTER ABSTRACTS| VOLUME 91. ISSUE 6, SUPPLEMENT, AB412, 2020.
14. Erinjeri J P. Clark T W. Cryoablation: mechanism of action and devices. J Vasc Interv Radiol. 2010 August: 21 (8 Suppl): S187-91. doi: 10.1016/j.jvir.2009.12.403. PMID: 20656228: PMCID: PMC6661161.
15. Yakkala C, Chiang C L. Kandalaft L, Denys A. Duran R. Cryoablation and Immunotherapy: An Enthralling Synergy to Confront the Tumors. Front Immunol. 2019 Sep. 24:10:2283.
16. Erinjeri J P, Clark T W. Cryoablation: mechanism of action and devices. J Vasc Interv Radiol. 2010 August: 21 (8 Suppl): S187-91. doi: 10.1016/j.jvir.2009.12.403.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Citation of a reference herein shall not be construed as an admission that such reference is prior art to the present invention. All references cited herein are hereby incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Although the present invention has been described for the purpose of illustration, it is understood that such detail is solely for that purpose and variations can be made by those skilled in the art without departing from the spirit and scope of the invention which is defined by the following claims.

What is claimed is:

1. A catheter comprising:
a probe having a proximal end, a distal end, and a body portion disposed between the proximal and distal ends; and
a cryogenic supply assembly housed within the probe, said cryogenic supply assembly comprising a sprayer orifice assembly and a cryogenic supply line within the sprayer orifice assembly,
wherein said sprayer orifice assembly comprises a sprayer tube and a seal chamber component, said sprayer tube comprising one or more sprayer orifices for delivery of a cryogenic fluid from the cryogenic supply line to the probe, and said seal chamber component comprises a seal chamber associated with each of the one or more sprayer orifices and forming a fluidic conduit between the cryogenic supply line and the one or more sprayer orifices through which the cryogenic fluid flows to the probe, and
wherein said cryogenic supply assembly is configured so that the cryogenic supply line and the sprayer tube move either axially independent of one another or axially together as one unit.

2. The catheter according to claim 1, wherein said sprayer tube comprises a plurality of sprayer orifices.

3. The catheter according to claim 2, wherein the plurality of sprayer orifices are arranged to include a proximal sprayer orifice, a distal sprayer orifice, and optionally one or more sprayer orifices located between the proximal and distal sprayer orifices.

4. The catheter according to claim 2, wherein the plurality of sprayer orifices are arranged so that they are located at either the same or different circumferential positions of the sprayer tube.

5. The catheter according to claim 1, wherein said sprayer orifice assembly further comprises an overtube disposed within the spray tube and around the cryogenic supply line.

6. The catheter according to claim 1, wherein said sprayer orifice assembly further comprises an inner support tube disposed inside of the sprayer tube, an outer support tube disposed outside of the sprayer tube, or both an inner support tube and an outer support tube.

7. The catheter according to claim 1, wherein said sprayer orifice assembly further comprises a restrictor component positioned over the one or more sprayer orifices and configured to cause the cryogenic fluid to exit the one or more sprayer orifices at a predetermined spray pattern, spray width, spray direction, traversing spray speed, and/or flow rate.

8. The catheter according to claim 7, wherein the restrictor component is selected from the group consisting of a mesh, braid, a hole or plurality of holes of various shapes and sizes, and a slit or plurality of slits of various dimensions.

9. The catheter according to claim 8, wherein the holes and slits are laser cut holes or slits of reinforced tube configured to expose a reinforcement portion of the reinforced tube.

10. The catheter according to claim 1, wherein said sprayer orifice assembly further comprises a spray guide associated with the one or more of the sprayer orifices to control spray pattern of the cryogenic fluid as it exits the one or more sprayer orifices.

11. The catheter according to claim 10, wherein the spray guide comprises a wall structure positioned above and encircling the one or more sprayer orifices, said wall structure extending externally from the spray tube at a desired width, wherein the spray guide is configured so that cryogenic fluid exiting from the sprayer orifice will have a spray pattern that is more constricted and/or precise as the width of the wall structure of the spray guide increases.

12. The catheter according to claim 1 further comprising a reinforcement tube disposed around the cryogenic supply line and abutted to the sprayer tube, thereby reducing (restricting) angulation of the sprayer caused by the flow of fluid during use or reducing (restricting) the distance the sprayer moves away from the inner surface of the probe during use.

13. The catheter according to claim 1, wherein said seal chamber component further comprises a seal stop component and an orifice seal component associated with each seal chamber.

14. The catheter according to claim 1, further comprising a distal probe tip attached to the distal end of the probe.

15. The catheter according to claim 1, wherein said catheter is configured for selective ablation in the mucosa and submucosa of a gastrointestinal tract of a subject.

16. The catheter according to claim 1, wherein the treatment tissue comprises mucosal and/or submucosal tissue of the large intestine, small intestine, stomach, esophagus, rectum, and anus.

17. A cryogenic ablation system comprising:
a catheter according to any one of claims 1-16; and
a controller configured to control the functionality of the catheter for delivering a cryogenic fluid to the cryogenic catheter probe for selective ablation in mucosa and submucosa of a gastrointestinal tract of a subject.

18. The system according to claim 17, wherein the system further comprises a shaft connected to the proximal end of the cryogenic catheter probe and/or running through all or a portion of the cryogenic catheter probe.

19. The system according to claim 18, wherein the shaft is connected to a handle.

20. The system according to claim 19, wherein the handle further comprises a high pressure plate.

21. The system according to claim 20, wherein the handle further comprises a hub-cap connected to the high pressure plate.

22. The system according to claim 17, wherein the system further comprises a pressure detection tube disposed within the cryogenic catheter probe or shaft.

23. The system according to claim 17, wherein the cryogenic catheter probe is placed into an expanded state upon release of cryogenic fluid into the inside of the cryogenic catheter probe.

24. The system according to claim 17, wherein the controller comprises one or more variable controller parameters used to control functional assembly.

25. The system according to claim 24, wherein the controller is configured to perform closed-loop energy delivery to the functional assembly based on the sensor signal.

26. The system according to claim 17, wherein the system further comprises at least one sensor constructed and arranged to produce a sensor signal.

27. A method of performing cryogenic ablation of mucosal tissue and/or of both mucosal tissue and submucosal tissue in the gastrointestinal tract of a subject, said method comprising:
(a) providing a cryogenic ablation system according to claim 17;
(b) contacting the cryogenic catheter probe of the system with a target treatment region of the gastrointestinal tract of the subject; and
(c) releasing a cryogenic fluid from the one or more sprayer orifices to treat the target treatment region by cryogenically ablating at least a portion the mucosal tissue or ablating at least a portion of both the mucosal and submucosal tissue of the target treatment region.

28. The method according to claim 27, wherein the target treatment region comprises mucosal tissue and/or both mucosal and submucosal tissue of the large intestine, small intestine, stomach, esophagus, rectum, or anus of the subject.

29. The method according to claim 28, wherein treating the target treatment region comprises performing a series of tissue ablation steps, each comprising ablation of an axial length of the large intestine, small intestine, stomach, esophagus, rectum, or anus of the subject, wherein each ablation step is optionally preceded by a tissue expansion step.

30. The method according to claim 27, further comprises adjusting at least one variable controller parameter based on the sensor signal.

31. Use of a cryogenic ablation system in a method of performing cryogenic ablation of mucosal tissue and/or of both mucosal tissue and submucosal tissue in the gastrointestinal tract of a subject, wherein said method comprises:
(a) providing a cryogenic ablation system according to claim 17;
(b) contacting the cryogenic catheter probe of the system with a target treatment region of the gastrointestinal tract of the subject; and
(c) releasing a cryogenic fluid from the one or more sprayer orifices to treat the target treatment region by cryogenically ablating at least a portion the mucosal tissue or ablating at least a portion of both the mucosal and submucosal tissue of the target treatment region.

32. A cryogenic ablation system for use in a method of performing cryogenic ablation of mucosal tissue and/or of both mucosal tissue and submucosal tissue in the gastrointestinal tract of a subject, wherein said method comprises:
(a) providing a cryogenic ablation system according to claim 17;
(b) contacting the cryogenic catheter probe of the system with a target treatment region of the gastrointestinal tract of the subject; and
(c) releasing a cryogenic fluid from the one or more sprayer orifices to treat the target treatment region by cryogenically ablating at least a portion the mucosal tissue or ablating at least a portion of both the mucosal and submucosal tissue of the target treatment region.

33. A method for performing a medical procedure in a small intestine and/or stomach of a patient in need of said medical procedure, the method comprising:
(a) providing a cryogenic ablation system according to claim 17;
(b) contacting the cryogenic catheter probe of the system with a target treatment region of the small intestine and/or stomach of the patient; and
(c) releasing a cryogenic fluid from the one or more sprayer orifices to treat the target treatment region by cryogenically ablating at least a portion the mucosal tissue or ablating at least a portion of both the mucosal and submucosal tissue of the target treatment region,
thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

34. The method according to claim 33, wherein treating the target treatment region comprises performing a series of tissue ablation steps, each comprising ablation of an axial length of the small intestine or stomach tissue, wherein each ablation step is optionally preceded by a tissue expansion step.

35. The method according to claim 33, further comprises adjusting at least one variable controller parameter based on the sensor signal.

36. Use of a cryogenic ablation system in a method for performing a medical procedure in a small intestine and/or stomach of a patient in need of said medical procedure, the method comprising:
(a) providing a cryogenic ablation system according to claim 17;
(b) contacting the cryogenic catheter probe of the system with a target treatment region of the small intestine and/or stomach of the patient; and
(c) releasing a cryogenic fluid from the one or more sprayer orifices to treat the target treatment region by cryogenically ablating at least a portion the mucosal tissue or ablating at least a portion of both the mucosal and submucosal tissue of the target treatment region,
thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.

37. A cryogenic ablation system for use in a method for performing a medical procedure in a small intestine and/or stomach of a patient in need of said medical procedure, the method comprising:
(a) providing a cryogenic ablation system according to claim 17;
(b) contacting the cryogenic catheter probe of the system with a target treatment region of the small intestine and/or stomach of the patient; and
(c) releasing a cryogenic fluid from the one or more sprayer orifices to treat the target treatment region by cryogenically ablating at least a portion the mucosal tissue or ablating at least a portion of both the mucosal and submucosal tissue of the target treatment region,
thereby performing a medical procedure to treat a condition of the patient selected from the group consisting of Type 1 diabetes, Type 2 diabetes, non-alcoholic fatty liver disease (NAFLD), non-alcoholic steatohepatitis (NASH), and obesity.
